(19) United States
(12) Patent Application Publication
(10) Pub. No.: US 2024/0158387 A1
(43) Pub. Date: May 16, 2024

(54) CRYSTALLINE FORMS, COMPOSITIONS CONTAINING SAME, AND METHODS OF THEIR USE

(71) Applicant: FOGHORN THERAPEUTICS INC., Cambridge, MA (US)

(72) Inventors: Rishi G. VASWANI, Lexington, MA (US); Chong-Hui GU, Waban, MA (US)

(21) Appl. No.: 18/281,022

(22) PCT Filed: Mar. 9, 2022
(86) PCT No.: PCT/US22/19506

§ 371 Date: Sep. 8, 2023

Related U.S. Application Data
(60) Provisional application No. 63/158,716, filed on Mar. 9, 2021.

(51) Int. Cl. C07D 417/14; A61K 9/48; A61K 45/06

(52) U.S. Cl. CPC C07D 417/14; A61K 9/4816; A61K 9/4858; A61K 9/4866; A61K 45/06; C07B 2200/13

(57) ABSTRACT

Disclosed are crystalline forms of the compound of formula (I): (I) Also disclosed are compositions containing the crystalline forms of the compound of formula (I) and methods of their use. The methods may be for treating cancer in a subject in need thereof, e.g., by administering to the subject an effective amount of the crystalline form disclosed herein or its composition.

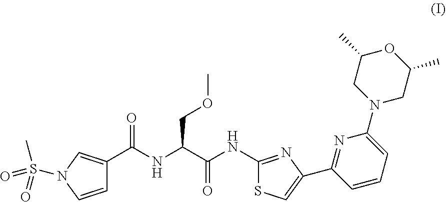 [SMILES: COC[C@H](NC(=O)c1ccn(S(C)(=O)=O)c1)C(=O)Nc1nc(-c2cccc(N3C[C@@H](C)O[C@@H](C)C3)n2)cs1]

### BACKGROUND

The invention relates to crystalline forms, compositions containing them, and methods of their use, e.g., in inhibiting the activity of a BAF complex in a subject or in treating cancer in a subject.

Chromatin regulation is essential for gene expression, and ATP-dependent chromatin remodeling is a mechanism by which such gene expression occurs. The human Switch/Sucrose Non-Fermentable (SWI/SNF) chromatin remodeling complex, also known as BAF complex, has two SWI2-like ATPases known as BRG1 (Brahma-related gene-1) and BRM (Brahma). The transcription activator BRG1, also known as ATP-dependent chromatin remodeler SMARCA4, is encoded by the SMARCA4 gene on chromosome 19. BRG1 is overexpressed in some cancer tumors and is needed for cancer cell proliferation. BRM, also known as probable global transcription activator SNF2L2 and/or ATP-dependent chromatin remodeler SMARCA2, is encoded by the SMARCA2 gene on chromosome 9 and has been shown to be essential for tumor cell growth in cells characterized by loss of BRG1 function mutations. Deactivation of BRG and/or BRM results in downstream effects in cells, including cell cycle arrest and tumor suppression.

### SUMMARY OF THE INVENTION

The present invention features crystalline forms of a compound useful for modulating a BAF complex.

In one aspect, the invention provides a crystalline form of the compound of formula (I):

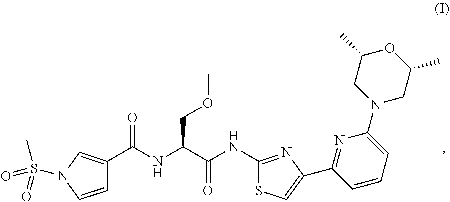 [SMILES: COC[C@H](NC(=O)c1ccn(S(C)(=O)=O)c1)C(=O)Nc1nc(-c2cccc(N3C[C@@H](C)O[C@@H](C)C3)n2)cs1]

where the crystalline form is characterized by a powder x-ray diffraction pattern having peaks at 14.4 °2θ±0.2 °2θ and 17.4 °2θ±0.2 °2θ. In some embodiments, the crystalline form is further characterized by a powder x-ray diffraction pattern having peaks at 11.7 °2θ±0.2 °2θ and 15.2 °2θ±0.2 °2θ. In some embodiments, the crystalline form is further characterized by a powder x-ray diffraction pattern having peaks at 14.9 °2θ±0.2 °2θ and 18.5 °2θ±0.2 °2θ. In some embodiments, the crystalline form is further characterized by a powder x-ray diffraction pattern having peaks at 16.0 °2θ±0.2 °2θ and 19.1 °2θ±0.2 °2θ. In some embodiments, the crystalline form is further characterized by a powder x-ray diffraction pattern having a peak at 16.3 °2θ±0.2 °2θ. In some embodiments, the crystalline form is further characterized by a powder x-ray diffraction pattern having peaks at 20.6 °2θ±0.2 °2θ and 26.3 °2θ±0.2 °2θ. In some embodiments, the crystalline form is further characterized by a powder x-ray diffraction pattern having peaks at 21.2 °2θ±0.2 °2θ and 26.6 °2θ±0.2 °2θ.

In some embodiments, the crystalline form is further characterized by a differential scanning calorimetry thermogram having an endothermic event onset at 173.5° C. to 180.1° C.

In another aspect, the invention provides a crystalline form of the compound of formula (I):

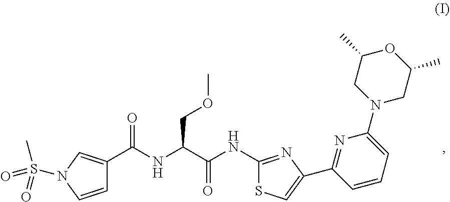 [SMILES: COC[C@H](NC(=O)c1ccn(S(C)(=O)=O)c1)C(=O)Nc1nc(-c2cccc(N3C[C@@H](C)O[C@@H](C)C3)n2)cs1]

where the crystalline form is characterized by a powder x-ray diffraction pattern having peaks at 7.7 °2θ±0.2 °2θ, 9.4 °2θ±0.2 °2θ and 17.0 °2θ±0.2 °2θ. In some embodiments, the crystalline form is further characterized by a powder x-ray diffraction pattern having peaks at 18.8 °2θ±0.2 °2θ and 20.0 °2θ±0.2 °2θ. In some embodiments, the crystalline form is further characterized by a powder x-ray diffraction pattern having a peak at 23.7 °2θ±0.2 °2θ. In some embodiments, the crystalline form is further characterized by a powder x-ray diffraction pattern having a peak at 14.8 °2θ±0.2 °2θ.

In some embodiments, the crystalline form is further characterized by a differential scanning calorimetry thermogram having an endothermic event onset at 196.0° C. to 198.8° C.

In yet another aspect, the invention provides a pharmaceutical composition including a crystalline form described herein.

In still another aspect, the invention provides a unit dosage form including the crystalline form described herein. In some embodiments, the unit dosage form is a capsule. In some embodiments, the unit dosage form includes one or more of a filler (e.g., microcrystalline cellulose, mannitol, or a combination thereof), a disintegrant (e.g., croscarmellose sodium), a wetting agent (e.g., sodium lauryl sulfate), a glidant (e.g., colloidal silicon dioxide), a lubricant (e.g., magnesium stearate), and a capsule shell.

In some embodiments, the unit dosage form includes 70 to 90% (w/w) of the filler. In some embodiments, the unit dosage form includes 4 to 6% (w/w) of the disintegrant. In some embodiments, the unit dosage form includes 0.5 to 1.5% (w/w) of the wetting agent. In some embodiments, the unit dosage form includes 1.5 to 2.5% (w/w) of the glidant. In some embodiments, the unit dosage form includes 0.4 to 0.6% (w/w) of the lubricant.

In some embodiments, the capsule shell includes a polymeric shell. In some embodiments, the polymeric shell includes hypromellose and titanium dioxide.

In a further aspect, the invention provides a method of decreasing the activity of a BAF complex in a subject, the method including administering to the subject an effective amount of the crystalline form described herein, the pharmaceutical composition described herein, or the unit dosage form described herein.

In yet further aspect, the invention provides a method of inhibiting BRM in a subject, the method including administering to the subject an effective amount of the crystalline form described herein, the pharmaceutical composition described herein, or the unit dosage form described herein.

In still further aspect, the invention provides a method of inhibiting BRG1 in a subject, the method including administering to the subject an effective amount of the crystalline form described herein, the pharmaceutical composition described herein, or the unit dosage form described herein.

In another aspect, the invention provides a method of inhibiting BRM and BRG1 in a subject, the method including administering to the subject an effective amount of the crystalline form described herein, the pharmaceutical composition described herein, or the unit dosage form described herein.

In yet another aspect, the invention provides a method of inducing apoptosis in a subject, the method including administering to the subject an effective amount of the crystalline form described herein, the pharmaceutical composition described herein, or the unit dosage form described herein.

In some embodiments, the subject has cancer.

In still another aspect, the invention provides a method of treating a BAF complex-related disorder in a subject in need thereof, the method including administering to the subject an effective amount of the crystalline form described herein, the pharmaceutical composition described herein, or the unit dosage form described herein.

In a further aspect, the invention provides a method of treating a disorder related to a BRG1 loss of function mutation in a subject in need thereof, the method including administering to the subject an effective amount of the crystalline form described herein, the pharmaceutical composition described herein, or the unit dosage form described herein.

In some embodiments, the subject is determined to have a BRG1 loss of function disorder. In some embodiments, the BAF complex-related disorder or disorder related to a BRG1 loss of function mutation is a cancer, a viral infection, coffin siris, neurofibromatosis (e.g., selected from the group consisting of neurofibromatosis type I (NF-1), neurofibromatosis type II (NF-2), and Schwannomatosis), or multiple meningioma.

In yet further aspect, the invention provides a method of treating cancer in a subject in need thereof, the method including administering to the subject an effective amount of the crystalline form described herein, the pharmaceutical composition described herein, or the unit dosage form described herein.

In still further aspect, the invention provides a method of reducing tumor growth of cancer in a subject in need thereof, the method including administering to the subject an effective amount of the crystalline form described herein, the pharmaceutical composition described herein, or the unit dosage form described herein.

In another aspect, the invention provides a method of suppressing metastatic progression of cancer in a subject in need thereof, the method including administering to the subject an effective amount of the crystalline form described herein, the pharmaceutical composition described herein, or the unit dosage form described herein.

In yet another aspect, the invention provides a method of suppressing metastatic colonization of cancer in a subject in need thereof, the method including administering to the subject an effective amount of the crystalline form described herein, the pharmaceutical composition described herein, or the unit dosage form described herein.

In still another aspect, the invention provides a method of reducing the level and/or activity of BRG1 and/or BRM in a cancer in a subject in need thereof, the method including administering to the subject an effective amount of the crystalline form described herein, the pharmaceutical composition described herein, or the unit dosage form described herein.

In some embodiments, the cancer is non-small cell lung cancer, colorectal cancer, bladder cancer, cancer of unknown primary, glioma, breast cancer, melanoma, non-melanoma skin cancer, endometrial cancer, esophageal cancer, esophagogastric cancer, pancreatic cancer, hepatobiliary cancer, soft tissue sarcoma, ovarian cancer, head and neck cancer, renal cell carcinoma, bone cancer, non-Hodgkin lymphoma, small-cell lung cancer, prostate cancer, embryonal tumor, germ cell tumor, cervical cancer, thyroid cancer, salivary gland cancer, gastrointestinal neuroendocrine tumor, uterine sarcoma, gastrointestinal stromal tumor, CNS cancer, thymic tumor, adrenocortical carcinoma, appendiceal cancer, small bowel cancer, penile cancer, bone cancer, or hematologic cancer. In some embodiments, the cancer is esophageal cancer.

In some embodiments, the cancer is non-small cell lung cancer, colorectal cancer, bladder cancer, cancer of unknown primary, glioma, breast cancer, melanoma, non-melanoma skin cancer, endometrial cancer, penile cancer, bone cancer, renal cell carcinoma, prostate cancer, or hematologic cancer. In some embodiments, the cancer is non-small cell lung cancer.

In some embodiments, the cancer is melanoma, prostate cancer, breast cancer, bone cancer, renal cell carcinoma, or hematologic cancer. In some embodiments, the cancer is melanoma (e.g., uveal melanoma, mucosal melanoma, or cutaneous melanoma). In some embodiments, the melanoma is uveal melanoma.

In some embodiments, the cancer is prostate cancer.

In some embodiments, the cancer is hematologic cancer (e.g., multiple myeloma, large cell lymphoma, acute T-cell leukemia, acute myeloid leukemia, myelodysplastic syndrome, immunoglobulin A lambda myeloma, diffuse mixed histiocytic and lymphocytic lymphoma, B-cell lymphoma, acute lymphoblastic leukemia, diffuse large cell lymphoma, or non-Hodgkin's lymphoma). In some embodiments, the hematologic cancer is acute myeloid leukemia.

In some embodiments, the cancer is breast cancer (e.g., an ER positive breast cancer, an ER negative breast cancer, triple positive breast cancer, or triple negative breast cancer).

In some embodiments, the cancer is a bone cancer (e.g., Ewing's sarcoma).

In some embodiments, the cancer is a renal cell carcinoma (e.g., a microphthalmia transcription factor family translocation renal cell carcinoma).

In some embodiments, the cancer is non-small cell lung cancer, prostate cancer, esophagogastric cancer, hematologic cancer, or melanoma. In some embodiments, the hematologic cancer is acute myeloid leukemia. In some embodiments, melanoma is uveal melanoma.

In some embodiments, the cancer expresses BRG1 and/or BRM protein.

In some embodiments, the subject or cancer has a BRG1 loss of function mutation. In some embodiments, the BRG1 loss of function mutation is in the ATPase catalytic domain of the protein. In other embodiments, the BRG1 loss of function mutation is a deletion at the C-terminus of BRG1.

In some embodiments, the cancer does not have, or has been determined not to have, an epidermal growth factor receptor mutation and/or an anaplastic lymphoma kinase driver mutation.

In some embodiments, the cancer has, or has been determined to have, a KRAS mutation, a mutation in GNAQ, a mutation in GNA11, a mutation in PLCB4, a mutation in CYSLTR2, a mutation in BAP1, a mutation in SF3B1, a mutation in EIF1AX, a TFE3 translocation, a TFEB translocation, a MITF translocation, an EZH2 mutation, a SUZ12 mutation, and/or an EED mutation.

In some embodiments, the cancer is metastatic.

In some embodiments, the cancer is resistant to, or failed to respond to prior treatment with, an anticancer therapy (e.g., a chemotherapeutic or cytotoxic agent (e.g., a mitogen-activated protein kinase (MEK) inhibitor and/or a protein kinase C (PKC) inhibitor), immunotherapy, surgery, radiotherapy, thermotherapy, or photocoagulation, or a combination thereof). In some embodiments, the cancer is resistant to, or failed to respond to prior treatment with a PKC inhibitor.

In some embodiments, the method further includes administering to the subject an anticancer therapy (e.g., a chemotherapeutic or cytotoxic agent, immunotherapy, surgery, radiotherapy, thermotherapy, or photocoagulation, or a combination thereof). In some embodiments, the anticancer therapy is surgery, a MEK inhibitor (e.g., selumetinib, binimetinib, or tametinib), and/or a PKC inhibitor (e.g., sotrastaurin or IDE196), or a combination thereof.

In a further aspect, the invention provides a method for treating a viral infection in a subject in need thereof, the method including administering to the subject an effective amount of the crystalline form described herein, the pharmaceutical composition described herein, or the unit dosage form described herein.

In some embodiments, the viral infection is an infection with a virus of the Retroviridae family, a virus of the Hepadnaviridae family, a virus of the Flaviviridae family, a virus of the Adenoviridae family, a virus of the Herpesviridae family, a virus of the Papillomaviridae family, a virus of the Parvoviridae family, a virus of the Polyomaviridae family, a virus of the Paramyxoviridae family, or a virus of the Togaviridae family.

In some embodiments, the effective amount of the crystalline form of the compound reduces the level and/or activity of BRG1 by at least 5% as compared to a reference. In some embodiments, the effective amount of the crystalline form of the compound reduces the level and/or activity of BRG1 by at least 5% as compared to a reference for at least 12 hours.

In some embodiments, the effective amount of the crystalline form of the compound reduces the level and/or activity of BRM by at least 5% as compared to a reference. In some embodiments, the effective amount of the crystalline form of the compound reduces the level and/or activity of BRM by at least 5% as compared to a reference for at least 12 hours.

### Definitions

In this application, unless otherwise clear from context, (i) the term “a” may be understood to mean “at least one”; (ii) the term “or” may be understood to mean “and/or”; and (iii) the terms “comprising” and “including” may be understood to encompass itemized components or steps whether presented by themselves or together with one or more additional components or steps.

As used herein, the terms “about” and “approximately” refer to a value that is within 10% above or below the value being described. For example, the term “about 5%” indicates a range of from 4.5 to 5.5 As used herein, the term “administration” refers to the administration of a composition (e.g., a compound or a preparation that includes a compound as described herein) to a subject or system.

Administration to an animal subject (e.g., to a human) may be by any appropriate route. For example, in some embodiments, administration may be bronchial (including by bronchial instillation), buccal, enteral, interdermal, intra-arterial, intradermal, intragastric, intramedullary, intramuscular, intranasal, intraperitoneal, intrathecal, intratumoral, intravenous, intraventricular, mucosal, nasal, oral, rectal, subcutaneous, sublingual, topical, tracheal (including by intratracheal instillation), transdermal, vaginal, and vitreal.

As used herein, the term “BAF complex” refers to the BRG1- or HBRM-associated factors complex in a human cell.

As used herein, the term “BAF complex-related disorder” refers to a disorder that is caused or affected by the level of activity of a BAF complex.

As used herein, the term “BRG1 loss of function mutation” refers to a mutation in BRG1 that leads to the protein having diminished activity (e.g., at least 1% reduction in BRG1 activity, for example 2%, 5%, 10%, 25%, 50%, or 100% reduction in BRG1 activity). Exemplary BRG1 loss of function mutations include, but are not limited to, a homozygous BRG1 mutation and a deletion at the C-terminus of BRG1.

As used herein, the term “BRG1 loss of function disorder” refers to a disorder (e.g., cancer) that exhibits a reduction in BRG1 activity (e.g., at least 1% reduction in BRG1 activity, for example 2%, 5%, 10%, 25%, 50%, or 100% reduction in BRG1 activity).

The term “cancer” refers to a condition caused by the proliferation of malignant neoplastic cells, such as tumors, neoplasms, carcinomas, sarcomas, leukemias, and lymphomas.

As used herein, a “combination therapy” or “administered in combination” means that two (or more) different agents or treatments are administered to a subject as part of a defined treatment regimen for a particular disease or condition. The treatment regimen defines the doses and periodicity of administration of each agent such that the effects of the separate agents on the subject overlap. In some embodiments, the delivery of the two or more agents is simultaneous or concurrent and the agents may be co-formulated. In some embodiments, the two or more agents are not co-formulated and are administered in a sequential manner as part of a prescribed regimen. In some embodiments, administration of two or more agents or treatments in combination is such that the reduction in a symptom, or other parameter related to the disorder is greater than what would be observed with one agent or treatment delivered alone or in the absence of the other. The effect of the two treatments can be partially additive, wholly additive, or greater than additive (e.g., synergistic). Sequential or substantially simultaneous administration of each therapeutic agent can be effected by any appropriate route including, but not limited to, oral routes, intravenous routes, intramuscular routes, and direct absorption through mucous membrane tissues. The therapeutic agents can be administered by the same route or by different routes. For example, a first therapeutic agent of the combination may be administered by intravenous injection while a second therapeutic agent of the combination may be administered orally.

By a “decreased level” or an “increased level” of a protein or RNA is meant a decrease or increase, respectively, in a protein or RNA level, as compared to a reference (e.g., a decrease or an increase by about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, about 100%, about 150%, about 200%, about 300%, about 400%, about 500%, or more; a decrease or an increase of more than about 10%, about 15%, about 20%, about 50%, about 75%, about 100%, or about 200%, as compared to a reference; a decrease or an increase by less than about 0.01-fold, about 0.02-fold, about 0.1-fold, about 0.3-fold, about 0.5-fold, about 0.8-fold, or less; or an increase by more than about 1.2-fold, about 1.4-fold, about 1.5-fold, about 1.8-fold, about 2.0-fold, about 3.0-fold, about 3.5-fold, about 4.5-fold, about 5.0-fold, about 10-fold, about 15-fold, about 20-fold, about 30-fold, about 40-fold, about 50-fold, about 100-fold, about 1000-fold, or more). A level of a protein may be expressed in mass/vol (e.g., g/dL, mg/mL, μg/mL, ng/mL) or percentage relative to total protein in a sample.

By “decreasing the activity of a BAF complex” is meant decreasing the level of an activity related to a BAF complex, or a related downstream effect. A non-limiting example of decreasing an activity of a BAF complex is Sox2 activation. The activity level of a BAF complex may be measured using any method known in the art, e.g., the methods described in Kadoch et al. Cell, 2013, 153, 71-85, the methods of which are herein incorporated by reference.

A cancer “determined to be drug resistant,” as used herein, refers to a cancer that is drug resistant, based on unresponsiveness or decreased responsiveness to a chemotherapeutic agent, or is predicted to be drug resistant based on a prognostic assay (e.g., a gene expression assay).

By a “drug resistant” is meant a cancer that does not respond, or exhibits a decreased response to, one or more chemotherapeutic agents (e.g., any agent described herein).

As used herein, the term “failed to respond to a prior therapy” or “refractory to a prior therapy,” refers to a cancer that progressed despite treatment with the therapy.

As used herein, the term “inhibiting BRM” and/or “inhibiting BRG1” refers to blocking or reducing the level or activity of the ATPase catalytic binding domain or the bromodomain of the protein. BRM and/or BRG1 inhibition may be determined using methods known in the art, e.g., a BRM and/or BRG1 ATPase assay, a Nano DSF assay, or a BRM and/or BRG1 Luciferase cell assay.

As used herein, the term “LXS196,” also known as IDE196, refers to the PKC inhibitor having the structure:

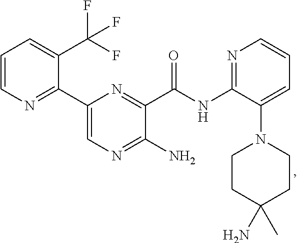 [SMILES: CC1(N)CCN(c2cccnc2NC(=O)c2nc(-c3ncccc3C(F)(F)F)cnc2N)CC1]

or a pharmaceutically acceptable salt thereof.

As used herein, “metastatic nodule” refers to an aggregation of tumor cells in the body at a site other than the site of the original tumor.

As used herein, “metastatic cancer” refers to a tumor or cancer in which the cancer cells forming the tumor have a high potential to or have begun to, metastasize, or spread from one location to another location or locations within a subject, via the lymphatic system or via haematogenous spread, for example, creating secondary tumors within the subject. Such metastatic behavior may be indicative of malignant tumors. In some cases, metastatic behavior may be associated with an increase in cell migration and/or invasion behavior of the tumor cells.

Examples of cancers that can be defined as metastatic include but are not limited to lung cancer (e.g., non-small cell lung cancer), breast cancer, ovarian cancer, colorectal cancer, biliary tract cancer, bladder cancer, brain cancer including glioblastomas and medullablastomas, cervical cancer, choriocarcinoma, endometrial cancer, esophageal cancer, gastric cancer, hematological neoplasms, multiple myeloma, leukemia, intraepithelial neoplasms, liver cancer, lymphomas, neuroblastomas, oral cancer, pancreatic cancer, prostate cancer, sarcoma, skin cancer including melanoma, basocellular cancer, squamous cell cancer, testicular cancer, stromal tumors, germ cell tumors, thyroid cancer, and renal cancer.

“Non-metastatic cell migration cancer” as used herein refers to cancers that do not migrate via the lymphatic system or via haematogenous spread.

The term “pharmaceutical composition,” as used herein, represents a composition containing a compound described herein formulated with a pharmaceutically acceptable excipient and appropriate for administration to a mammal, for example a human. Typically, a pharmaceutical composition is manufactured or sold with the approval of a governmental regulatory agency as part of a therapeutic regimen for the treatment of disease in a mammal. Pharmaceutical compositions can be formulated, for example, for oral administration in unit dosage form (e.g., a tablet, capsule, caplet, gelcap, or syrup); for topical administration (e.g., as a cream, gel, lotion, or ointment); for intravenous administration (e.g., as a sterile solution free of particulate emboli and in a solvent system suitable for intravenous use); or in any other pharmaceutically acceptable formulation.

A “pharmaceutically acceptable excipient,” as used herein, refers to any ingredient other than the compounds described herein (for example, a vehicle capable of suspending or dissolving the active compound) and having the properties of being substantially nontoxic and non-inflammatory in a patient. Excipients may include, for example: antiadherents, antioxidants, binders, coatings, compression aids, disintegrants, dyes (colors), emollients, emulsifiers, fillers (diluents), film formers or coatings, flavors, fragrances, glidants (flow enhancers), lubricants, preservatives, printing inks, sorbents, suspensing or dispersing agents, sweeteners, and waters of hydration.

“Proliferation” as used in this application involves reproduction or multiplication of similar forms (cells) due to constituting (cellular) elements.

By a “reference” is meant any useful reference used to compare protein or RNA levels. The reference can be any sample, standard, standard curve, or level that is used for comparison purposes. The reference can be a normal reference sample or a reference standard or level. A “reference sample” can be, for example, a control, e.g., a predetermined negative control value such as a “normal control” or a prior sample taken from the same subject; a sample from a normal healthy subject, such as a normal cell or normal tissue; a sample (e.g., a cell or tissue) from a subject not having a disease; a sample from a subject that is diagnosed with a disease, but not yet treated with a compound of the invention; a sample from a subject that has been treated by a compound of the invention; or a sample of a purified protein or RNA (e.g., any described herein) at a known normal concentration. By “reference standard or level” is meant a value or number derived from a reference sample. A “normal control value” is a pre-determined value indicative of non-disease state, e.g., a value expected in a healthy control subject. Typically, a normal control value is expressed as a range (“between X and Y”), a high threshold (“no higher than X”), or a low threshold (“no lower than X”). A subject having a measured value within the normal control value for a particular biomarker is typically referred to as “within normal limits” for that biomarker. A normal reference standard or level can be a value or number derived from a normal subject not having a disease or disorder (e.g., cancer); a subject that has been treated with a compound of the invention. In preferred embodiments, the reference sample, standard, or level is matched to the sample subject sample by at least one of the following criteria: age, weight, sex, disease stage, and overall health. A standard curve of levels of a purified protein or RNA, e.g., any described herein, within the normal reference range can also be used as a reference.

As used herein, the term “subject” refers to any organism to which a composition in accordance with the invention may be administered, e.g., for experimental, diagnostic, prophylactic, and/or therapeutic purposes. Typical subjects include any animal (e.g., mammals such as mice, rats, rabbits, non-human primates, and humans). A subject may seek or be in need of treatment, require treatment, be receiving treatment, be receiving treatment in the future, or be a human or animal who is under care by a trained professional for a particular disease or condition.

As used herein, the terms “treat,” “treated,” or “treating” mean therapeutic treatment or any measures whose object is to slow down (lessen) an undesired physiological condition, disorder, or disease, or obtain beneficial or desired clinical results. Beneficial or desired clinical results include, but are not limited to, alleviation of symptoms; diminishment of the extent of a condition, disorder, or disease; stabilized (i.e., not worsening) state of condition, disorder, or disease; delay in onset or slowing of condition, disorder, or disease progression; amelioration of the condition, disorder, or disease state or remission (whether partial or total); an amelioration of at least one measurable physical parameter, not necessarily discernible by the patient; or enhancement or improvement of condition, disorder, or disease. Treatment includes eliciting a clinically significant response without excessive levels of side effects. Treatment also includes prolonging survival as compared to expected survival if not receiving treatment. Compounds of the invention may also be used to “prophylactically treat” or “prevent” a disorder, for example, in a subject at increased risk of developing the disorder.

Unless otherwise defined, all technical and scientific terms used herein have the same meaning as commonly understood by one of ordinary skill in the art to which this invention belongs. Methods and materials are described herein for use in the present disclosure; other, suitable methods and materials known in the art can also be used. The materials, methods, and examples are illustrative only and not intended to be limiting. All publications, patent applications, patents, sequences, database entries, and other references mentioned herein are incorporated by reference in their entirety. In case of conflict, the present specification, including definitions, will control.

The details of one or more embodiments of the invention are set forth in the description below. Other features, objects, and advantages of the invention will be apparent from the description and from the claims.

### BRIEF DESCRIPTION OF THE DRAWINGS

FIGS. 1A and 1B are a series of microscopic images of the first and second crystalline forms, respectively, of the compound of formula (I). The images were made in polarized, visible light.

FIG. 2A is a plot showing TG and DSC traces for the first crystalline form of the compound of formula (I). The TG trace exhibited low temperature weight loss of 0.3% (w/w) due to loss of the free water in the sample. The DSC trace displayed a sharp endothermic event (onset at about 173.5° C.) which was likely due to the sample melting.

FIG. 2B is a plot showing TG and DSC traces for the second crystalline form of the compound of formula (I). The TG trace showed no significant loss in mass prior melting, with a weight loss of 0.4% (w/w), indicating the inclusion of solvent in the crystal structure. The DSC trace displayed a sharp endothermic event (onset at about 196.0° C.) which was likely due to the sample melting.

FIG. 3 is a plot showing modulated DSC traces for the first crystalline form of the compound of formula (I) with two heating cycles.

FIG. 4A is a plot showing the DVS cycles for the first crystalline form of the compound of formula (I). The DVS analysis showed at total of 1.4% mass increase up to 80% RH. The material was thus slightly hygroscopic.

FIG. 4B is a DVS kinetic plot for the first crystalline form of the compound of formula (I).

FIG. 5A is a plot showing the DVS cycles for the second crystalline form of the compound of formula (I). The DVS analysis showed at total of 0.04% mass increase up to 80% RH. The material was non-hygroscopic.

FIG. 5B is a DVS kinetic plot for the second crystalline form of the compound of formula (I).

FIG. 6 is a XRPD pattern of the first crystalline form of the compound of formula (I).

FIG. 7 is a XRPD pattern of the second crystalline form of the compound of formula (I).

FIG. 8 is an overlay of the XRPD pattern of the first crystalline form of the compound of formula (I) (trace in black) and the XRPD pattern of the first crystalline form of the compound of formula (I) after exposure to high humidity after DVS analysis (trace in magenta).

FIG. 9 is an overlay of the XRPD pattern of the second crystalline form of the compound of formula (I) (trace in black) and the XRPD pattern of the second crystalline form of the compound of formula (I) after exposure to high humidity after DVS analysis (trace in blue).

FIG. 10 is an ¹H NMR spectra of the second crystalline form of the compound of formula (I).

FIG. 11 is an overlay of the XRPD patterns obtained from solid samples isolated from single solvent slurries of the second crystalline form of the compound of formula (I) (traces from top to bottom: solid from ethyl acetate, solid from methyl tert-butyl ether, solid from acetonitrile, solid from methyl ethyl ketone, solid from acetone, solid from isopropanol, solid from ethanol, solid from methanol, the second crystalline form of the compound of formula (I), solid from xylenes, solid from 2-butanol, solid from heptane, solid from water, solid from toluene, solid from 2-methyl tetrahydrofuran, solid from isopropyl acetate, and the second crystalline form of the compound of formula (I)). Each diffraction pattern displays the characteristic reflections of the second crystalline form of the compound of formula (I), with reflections at 7.5 °2θ, 9.5 °2θ, 15.0 °2θ, and 17.0 °2θ.

FIG. 12 is an overlay of the XRPD patterns obtained from solid samples isolated from temperature cycled single solvent slurries of the second crystalline form of the compound of formula (I) (traces from top to bottom: solid from ethyl acetate, solid from isopropyl acetate, solid from 2-methyl tetrahydrofuran, solid from toluene, solid from xylenes, solid from water, solid from heptane, the second crystalline form of the compound of formula (I), solid from methyl tert-butyl ether, solid from acetonitrile, solid from methyl ethyl ketone, solid from acetone, solid from 2-butanol, solid from isopropanol, solid from ethanol, solid from methanol, and the second crystalline form of the compound of formula (I)). Each diffraction pattern displays the characteristic reflections of the second crystalline form of the compound of formula (I), with reflections at 7.5 °2θ, 9.5 °2θ, 15.0 °2θ, and 17.0 °2θ, although samples obtained from 2-butanol and xylenes exhibited poor crystallinity.

FIG. 13 is an overlay of the XRPD patterns obtained from solid samples isolated from a low temperature evaporation screen of the second crystalline form of the compound of formula (I) in various solvents, generated by slow solvent evaporation under ambient conditions (traces from top to bottom: solid from isopropyl acetate, solid from ethyl acetate, solid from acetonitrile, solid from methyl ethyl ketone, solid from acetone, solid from 2-butanol, solid from isopropanol, solid from ethanol, solid from methanol, and the second crystalline form of the compound of formula (I)). Each diffraction pattern displays the characteristic reflections of the second crystalline form of the compound of formula (I), with reflections at 7.5 °2θ, 9.5 029, 15.0 °2θ, and 17.0 °2θ, although samples obtained from tetrahydrofuran and 2-methyl tetrahydrofuran resulted in an oil.

FIG. 14 is an overlay of the XRPD patterns obtained from solid samples isolated from a high temperature evaporation screen of the second crystalline form of the compound of formula (I) in various solvents (traces from top to bottom: solid from toluene, solid from 2-methyl tetrahydrofuran, solid from isopropyl acetate, solid from ethyl acetate, solid from acetonitrile, solid from methyl ethyl ketone, solid from acetone, solid from isopropanol, solid from methanol, and the second crystalline form of the compound of formula (I)). The liquors were heated from 45° C. to 50° C. to ensure all solids were fully dissolved before the solvent was evaporated at 50° C. XRPD analysis showed that material produced by the evaporation of isopropanol, isopropyl acetate, and toluene formed the second crystalline form of the compound of formula (I). All other solid samples were amorphous.

FIG. 15 is an overlay of the XRPD patterns obtained from solid samples isolated by crash cooling in ethyl acetate, isopropanol, methyl ethyl ketone, ethanol, methanol, and acetonitrile (traces from top to bottom, with the bottom trace being the second crystalline form of the compound of formula (I)). The XRPD patterns are consistent with the second crystalline form of the compound of formula (I).

FIG. 16 is an overlay of the XRPD patterns obtained from solid samples isolated after a hydrate screen (traces from top to bottom: solid from tetrahydrofuran/water, solid from acetone/water, solid from acetonitrile/water, solid from isopropanol/water, solid from ethanol/water, solid from methanol/water, the second crystalline form of the compound of formula (I), and the first crystalline form of the compound of formula (I)). The XRPD patterns were performed on wet samples to ensure any alterations resulting from drying were observed. The XRPD patterns are consistent with the second crystalline form of the compound of formula (I), except the XRPD pattern of the solid sample from tetrahydrofuran/water, which is amorphous.

FIG. 17 is an overlay of the XRPD patterns performed on wet and dry samples of the solid obtained from a hydrate screen with methanol/water with the XRPD patterns of the first and second crystalline forms of the compound of formula (I) (black and magenta traces, respectively). The XRPD pattern of the wet solid obtained from methanol/water (trace in blue), which exhibited an additional reflection at 11.5 °2θ, corresponding with the first crystalline form of the compound of formula (I). The dry solid (trace in green) exhibited only peaks corresponding with the second crystalline form of the compound of formula (I).

FIG. 18 is an overlay of the XRPD patterns obtained from solid samples stirred in acetonitrile at 60° C., acetonitrile at room temperature, methanol at 60° C., methanol at room temperature, ethyl acetate at 60° C., and ethyl acetate at room temperature. The bottom traces are the first and second crystalline forms of the compound of formula (I) (black and magenta traces, respectively).

FIG. 19 is an overlay of the XRPD patterns of solids stirred for 14 days in methanol/water (green trace: solid from methanol/water, room temperature; blue trace: solid from methanol/water, 60° C.; black trace: the first crystalline form of the compound of formula (I); magenta trace: the second crystalline form of the compound of formula (I)). These samples showed a majority of the second crystalline form, with reflections characteristic of the first crystalline form visible at 14.4 °2θ and 17.4 °2θ.

FIG. 20 is an overlay of DSC traces of the first and second crystalline forms (black and magenta traces, respectively) of the compound of formula (I) with samples stirred in methanol/water at room temperature and 60° C. (blue and green traces, respectively).

FIG. 21 is an overlay of the XRPD patterns of solids stirred for 7 days in acetonitrile/water (green trace: solid from acetonitrile/water, 60° C.; blue trace: solid from acetonitrile/water, room temperature; black trace: the first crystalline form of the compound of formula (I); magenta trace: the second crystalline form of the compound of formula (I)).

FIG. 22 is an overlay of DSC traces of the first and second crystalline forms (black and magenta traces, respectively) of the compound of formula (I) with samples stirred in acetonitrile/water at room temperature and 60° C. (blue and green traces, respectively).

FIG. 23 is an overlay of XRPD patterns of the second crystalline form of the compound of formula (I) (black trace), magnesium sulfate (magenta trace), and the magnesium sulfate salt of the compound of formula (I) (blue trace).

FIG. 24 is an overlay of ¹H NMR spectra of the second crystalline form of the compound of formula (I) (blue trace) and the magnesium sulfate salt of the compound of formula (I) (magenta trace).

FIG. 25 is an overlay of HPLC traces of the compound of formula (I) (top trace) and the magnesium sulfate salt (bottom trace).

FIG. 26 is a plot showing TG and DSC traces for the magnesium sulfate salt of the compound of formula (I). The TG trace exhibited a significant low temperature weight loss of 11% (w/w) with a corresponding low temperature endotherm in the DSC trace suggesting solvent evaporation. A melting endotherm was observed at 115° C., followed by degradation from 165° C.

FIG. 27 is an overlay of XPRD patterns obtained from solid samples reacted with sodium hydroxide (blue trace) and potassium hydroxide (green trace) (the black trace is the second crystalline form of the compound of formula (I), and the magenta trace is sodium hydroxide). The new peaks in the blue and green traces represent new crystal structures; however, molecular degradation was visible via ¹H NMR spectroscopy.

FIG. 28 is an overlay of ¹H NMR spectra of the second crystalline form of the compound of formula (I) (blue trace) and materials formed in combining the compound of formula (I) with sodium hydroxide (magenta trace) and potassium hydroxide (green trace).

FIG. 29 is an overlay of HPLC chromatograms of the compound of formula (I) (bottom trace), and the materials formed in combining the compound of formula (I) with sodium hydroxide (middle trace) and potassium hydroxide (top trace). New peaks at 14.7 minutes in the sodium hydroxide and potassium hydroxide traces represent a degradation product.

FIG. 30 is an overlay of XPRD patterns of the second crystalline form of the compound of formula (I) (black trace), serine (magenta trace), material formed by the evaporation of isopropanol with the compound of formula (I) and serine (blue trace), and material formed by the evaporation of methyl tert-butyl ether with the compound of formula (I) and serine (green trace). The green and blue traces show previously unobserved reflections at low intensity, suggesting the early stages of formation of a new crystalline form.

FIG. 31 is an overlay of DSC traces, comparing serine (bottom trace) with the material formed by the evaporation of methyl tert-butyl ether with the compound of formula (I) and serine (top trace). The endotherm at 217° C. corresponds with the serine melting temperature and the endotherm at 227° C. may indicate the presence of a second structure.

FIG. 32 is an overlay of XRPD patterns obtained from HCl in ethyl acetate (blue trace), magnesium sulfate in toluene (green trace), and lysine in xylenes (purple trace). The black and red traces are references of the second crystalline form of the compound of formula (I) and lysine, respectively. XRPD analysis showed the HCl material was amorphous, the magnesium sulfate material showed reflections characteristic of the second crystal form of the compound of formula (I) only, and the lysine material showed lysine reflections

FIG. 33 is an overlay of XRPD patterns of hydrochloride salts formed in ethyl acetate (black trace), isopropanol (red trace), and isopropyl acetate (blue trace).

FIG. 34 is an overlay of ¹H NMR spectra of the compound of formula (I) (blue trace) with the hydrochloride salt of the compound of formula (I) (magenta trace).

FIG. 35 is a plot of the TGA and DSC of the hydrochloride salt of the compound of formula (I) formed in ethyl acetate. The sample formed in ethyl acetate exhibited weight loss of 3.7% (w/w) at approximately 150° C., and molecular degradation from 195° C. with onset at 235° C. A melting endotherm was observed at 182° C. with an onset at 163.2° C., followed by degradation.

FIG. 36A is a plot showing the DVS cycles for the hydrochloride salt of the compound of formula (I). DVS analysis showed a weight increase of 3.6% (w/w) during exposure to 80% RH, hence the material is classified as hygroscopic, with a total weight increase of 25.2% (w/w) during exposure to 95% RH.

FIG. 36B is a DVS kinetic plot for the hydrochloride salt of the compound of formula (I).

FIG. 37 is an overlay of ¹H NMR spectra of various salts of the compound of formula (I) formed in isopropyl acetate (traces from top to bottom: ethane disulfonic acid salt, tosylate salt, besylate salt, and the compound of formula (I)).

### DETAILED DESCRIPTION

In general, the invention provides crystalline forms of the compound of formula (I):

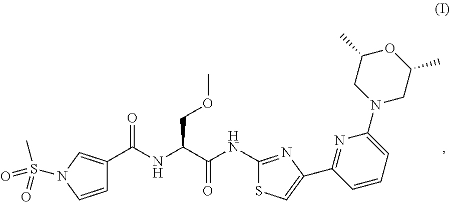 [SMILES: COC[C@H](NC(=O)c1ccn(S(C)(=O)=O)c1)C(=O)Nc1nc(-c2cccc(N3C[C@@H](C)O[C@@H](C)C3)n2)cs1]

In particular, the invention provides two crystalline forms of the compound of formula (I): a first crystalline form and a second crystalline form.

### Methods

A crystalline form described herein (e.g., the first or second crystalline form of the compound of formula (I)) is useful in the methods of the invention and, while not bound by theory, are believed to exert their ability to modulate the level, status, and/or activity of a BAF complex, i.e., by inhibiting the activity of the BRG1 and/or BRM proteins within the BAF complex in a mammal. BAF complex-related disorders include, but are not limited to, BRG1 loss of function mutation-related disorders.

An aspect of the present invention relates to methods of treating disorders related to BRG1 loss of function mutations such as cancer (e.g., non-small cell lung cancer, colorectal cancer, bladder cancer, cancer of unknown primary, glioma, breast cancer, melanoma, non-melanoma skin cancer, endometrial cancer, or penile cancer) in a subject in need thereof. In some embodiments, the present invention relates to methods of treating melanoma (e.g., uveal melanoma), prostate cancer, breast cancer, bone cancer, renal cell carcinoma, or a hematologic cancer.

In some embodiments, the compound is administered in an amount and for a time effective to result in one or more (e.g., two or more, three or more, four or more) of: (a) reduced tumor size, (b) reduced rate of tumor growth, (c) increased tumor cell death (d) reduced tumor progression, (e) reduced number of metastases, (f) reduced rate of metastasis, (g) decreased tumor recurrence (h) increased survival of subject, (i) increased progression free survival of subject.

Treating cancer can result in a reduction in size or volume of a tumor. For example, after treatment, tumor size is reduced by 5% or greater (e.g., 10%, 20%, 30%, 40%, 50%, 60%, 70%, 80%, 90%, or greater) relative to its size prior to treatment. Size of a tumor may be measured by any reproducible means of measurement. For example, the size of a tumor may be measured as a diameter of the tumor.

Treating cancer may further result in a decrease in number of tumors. For example, after treatment, tumor number is reduced by 5% or greater (e.g., 10%, 20%, 30%, 40%, 50%, 60%, 70%, 80%, 90%, or greater) relative to number prior to treatment. Number of tumors may be measured by any reproducible means of measurement, e.g., the number of tumors may be measured by counting tumors visible to the naked eye or at a specified magnification (e.g., 2×, 3×, 4×, 5×, 10×, or 50×).

Treating cancer can result in a decrease in number of metastatic nodules in other tissues or organs distant from the primary tumor site. For example, after treatment, the number of metastatic nodules is reduced by 5% or greater (e.g., 10%, 20%, 30%, 40%, 50%, 60%, 70%, 80%, 90% or greater) relative to number prior to treatment. The number of metastatic nodules may be measured by any reproducible means of measurement. For example, the number of metastatic nodules may be measured by counting metastatic nodules visible to the naked eye or at a specified magnification (e.g., 2×, 10×, or 50×).

Treating cancer can result in an increase in average survival time of a population of subjects treated according to the present invention in comparison to a population of untreated subjects. For example, the average survival time is increased by more than 30 days (more than 60 days, 90 days, or 120 days). An increase in average survival time of a population may be measured by any reproducible means. An increase in average survival time of a population may be measured, for example, by calculating for a population the average length of survival following initiation of treatment with the compound of the invention. An increase in average survival time of a population may also be measured, for example, by calculating for a population the average length of survival following completion of a first round of treatment with a pharmaceutically acceptable salt of the invention.

Treating cancer can also result in a decrease in the mortality rate of a population of treated subjects in comparison to an untreated population. For example, the mortality rate is decreased by more than 2% (e.g., more than 5%, 10%, or 25%). A decrease in the mortality rate of a population of treated subjects may be measured by any reproducible means, for example, by calculating for a population the average number of disease-related deaths per unit time following initiation of treatment with a pharmaceutically acceptable salt of the invention. A decrease in the mortality rate of a population may also be measured, for example, by calculating for a population the average number of disease-related deaths per unit time following completion of a first round of treatment with a pharmaceutically acceptable salt of the invention.

Exemplary cancers that may be treated by the invention include, but are not limited to, non-small cell lung cancer, small-cell lung cancer, colorectal cancer, bladder cancer, glioma, breast cancer, melanoma, non-melanoma skin cancer, endometrial cancer, esophagogastric cancer, pancreatic cancer, hepatobiliary cancer, soft tissue sarcoma, ovarian cancer, head and neck cancer, renal cell carcinoma, bone cancer, non-Hodgkin lymphoma, prostate cancer, embryonal tumor, germ cell tumor, cervical cancer, thyroid cancer, salivary gland cancer, gastrointestinal neuroendocrine tumor, uterine sarcoma, gastrointestinal stromal tumor, CNS cancer, thymic tumor, Adrenocortical carcinoma, appendiceal cancer, small bowel cancer, hematologic cancer, and penile cancer.

### Combination Formulations and Uses Thereof

The compounds of the invention can be combined with one or more therapeutic agents. In particular, the therapeutic agent can be one that treats or prophylactically treats any cancer described herein.

### Combination Therapies

A compound of the invention can be used alone or in combination with an additional therapeutic agent, e.g., other agents that treat cancer or symptoms associated therewith, or in combination with other types of treatment to treat cancer. In combination treatments, the dosages of one or more of the therapeutic compounds may be reduced from standard dosages when administered alone. For example, doses may be determined empirically from drug combinations and permutations or may be deduced by isobolographic analysis (e.g., Black et al., Neurology 65:S3-S6, 2005). In this case, dosages of the compounds when combined should provide a therapeutic effect.

In some embodiments, the second therapeutic agent is a chemotherapeutic agent (e.g., a cytotoxic agent or other chemical compound useful in the treatment of cancer). These include alkylating agents, antimetabolites, folic acid analogs, pyrimidine analogs, purine analogs and related inhibitors, vinca alkaloids, epipodopyyllotoxins, antibiotics, L-Asparaginase, topoisomerase inhibitors, interferons, platinum coordination complexes, anthracenedione substituted urea, methyl hydrazine derivatives, adrenocortical suppressant, adrenocorticosteroides, progestins, estrogens, antiestrogen, androgens, antiandrogen, and gonadotropin-releasing hormone analog. Also included is 5-fluorouracil (5-FU), leucovorin (LV), irenotecan, oxaliplatin, capecitabine, paclitaxel and doxetaxel. Non-limiting examples of chemotherapeutic agents include alkylating agents such as thiotepa and cyclosphosphamide; alkyl sulfonates such as busulfan, improsulfan and piposulfan; aziridines such as benzodopa, carboquone, meturedopa, and uredopa; ethylenimines and methylamelamines including altretamine, triethylenemelamine, trietylenephosphoramide, triethiylenethiophosphoramide and trimethylolomelamine; acetogenins (especially bullatacin and bullatacinone); a camptothecin (including the synthetic analogue topotecan); bryostatin; callystatin; CC-1065 (including its adozelesin, carzelesin and bizelesin synthetic analogues); cryptophycins (particularly cryptophycin 1 and cryptophycin 8); dolastatin; duocarmycin (including the synthetic analogues, KW-2189 and CB1-TM1); eleutherobin; pancratistatin; a sarcodictyin; spongistatin; nitrogen mustards such as chlorambucil, chlornaphazine, cholophosphamide, estramustine, ifosfamide, mechlorethamine, mechlorethamine oxide hydrochloride, melphalan, novembichin, phenesterine, prednimustine, trofosfamide, uracil mustard; nitrosureas such as carmustine, chlorozotocin, fotemustine, lomustine, nimustine, and ranimnustine; antibiotics such as the enediyne antibiotics (e.g., calicheamicin, especially calicheamicin gammall and calicheamicin omegall (see, e.g., Agnew, Chem. Intl. Ed Engl. 33:183-186 (1994)); dynemicin, including dynemicin A; bisphosphonates, such as clodronate; an esperamicin; as well as neocarzinostatin chromophore and related chromoprotein enediyne antiobiotic chromophores), aclacinomysins, actinomycin, authramycin, azaserine, bleomycins, cactinomycin, carabicin, caminomycin, carzinophilin, chromomycinis, dactinomycin, daunorubicin, detorubicin, 6-diazo-5-oxo-L-norleucine, Adriamycin® (doxorubicin, including morpholino-doxorubicin, cyanomorpholino-doxorubicin, 2-pyrrolino-doxorubicin and deoxydoxorubicin), epirubicin, esorubicin, idarubicin, marcellomycin, mitomycins such as mitomycin C, mycophenolic acid, nogalamycin, olivomycins, peplomycin, potfiromycin, puromycin, quelamycin, rodorubicin, streptonigrin, streptozocin, tubercidin, ubenimex, zinostatin, zorubicin; anti-metabolites such as methotrexate and 5-fluorouracil (5-FU); folic acid analogues such as denopterin, methotrexate, pteropterin, trimetrexate; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine, floxuridine; androgens such as calusterone, dromostanolone propionate, epitiostanol, mepitiostane, testolactone; anti-adrenals such as aminoglutethimide, mitotane, trilostane; folic acid replenisher such as frolinic acid; aceglatone; aldophosphamide glycoside; aminolevulinic acid; eniluracil; amsacrine; bestrabucil; bisantrene; edatraxate; defofamine; demecolcine; diaziquone; elfomithine; elliptinium acetate; an epothilone; etoglucid; gallium nitrate; hydroxyurea; lentinan; lonidainine; maytansinoids such as maytansine and ansamitocins; mitoguazone; mitoxantrone; mopidanmol; nitraerine; pentostatin; phenamet; pirarubicin; losoxantrone; podophyllinic acid; 2-ethylhydrazide; procarbazine; PSK® polysaccharide complex (JHS Natural Products, Eugene, Oreg.); razoxane; rhizoxin; sizofuran; spirogermanium; tenuazonic acid; triaziquone; 2,2′,2″-trichlorotriethylamine; trichothecenes (especially T-2 toxin, verracurin A, roridin A and anguidine); urethan; vindesine; dacarbazine; mannomustine; mitobronitol; mitolactol; pipobroman; gacytosine; arabinoside (“Ara-C”); cyclophosphamide; thiotepa; taxoids, e.g., Taxol® paclitaxel (Bristol-Myers Squibb Oncology, Princeton, N.J.), ABraxane®, cremophor-free, albumin-engineered nanoparticle formulation of paclitaxel (American Pharmaceutical Partners, Schaumberg, III.), and Taxotere® doxetaxel (Rhone-Poulenc Rorer, Antony, France); chloranbucil; Gemzar® gemcitabine; 6-thioguanine; mercaptopurine; methotrexate; platinum coordination complexes such as cisplatin, oxaliplatin and carboplatin; vinblastine; platinum; etoposide (VP-16); ifosfamide; mitoxantrone; vincristine; Navelbine® vinorelbine; novantrone; teniposide; edatrexate; daunomycin; aminopterin; xeloda; ibandronate; irinotecan (e.g., CPT-11); topoisomerase inhibitor RFS 2000; difluoromethylornithine (DMFO); retinoids such as retinoic acid; capecitabine; and pharmaceutically acceptable salts, acids or derivatives of any of the above. Two or more chemotherapeutic agents can be used in a cocktail to be administered in combination with the first therapeutic agent described herein. Suitable dosing regimens of combination chemotherapies are known in the art and described in, for example, Saltz et al. (1999) Proc ASCO 18:233a and Douillard et al. (2000) Lancet 355:1041-7.

In some embodiments, the second therapeutic agent is a therapeutic agent which is a biologic such a cytokine (e.g., interferon or an interleukin (e.g., IL-2)) used in cancer treatment. In some embodiments the biologic is an anti-angiogenic agent, such as an anti-VEGF agent, e.g., bevacizumab (Avastin®). In some embodiments the biologic is an immunoglobulin-based biologic, e.g., a monoclonal antibody (e.g., a humanized antibody, a fully human antibody, an Fc fusion protein or a functional fragment thereof) that agonizes a target to stimulate an anti-cancer response, or antagonizes an antigen important for cancer. Such agents include Rituxan (Rituximab); Zenapax (Daclizumab); Simulect (Basiliximab); Synagis (Palivizumab); Remicade (Infliximab); Herceptin (Trastuzumab); Mylotarg (Gemtuzumab ozogamicin); Campath (Alemtuzumab); Zevalin (lbritumomab tiuxetan); Humira (Adalimumab); Xolair (Omalizumab); Bexxar (Tositumomab-I-131); Raptiva (Efalizumab); Erbitux (Cetuximab); Avastin (Bevacizumab); Tysabri (Natalizumab); Actemra (Tocilizumab); Vectibix (Panitumumab); Lucentis (Ranibizumab); Soliris (Eculizumab); Cimzia (Certolizumab pegol); Simponi (Golimumab); Ilaris (Canakinumab); Stelara (Ustekinumab); Arzerra (Ofatumumab); Prolia (Denosumab); Numax (Motavizumab); ABThrax (Raxibacumab); Benlysta (Belimumab); Yervoy (Ipilimumab); Adcetris (Brentuximab Vedotin); Perjeta (Pertuzumab); Kadcyla (Ado-trastuzumab emtansine); and Gazyva (Obinutuzumab). Also included are antibody-drug conjugates.

The second agent may be a therapeutic agent which is a non-drug treatment. For example, the second therapeutic agent is radiation therapy, cryotherapy, hyperthermia and/or surgical excision of tumor tissue.

The second agent may be a checkpoint inhibitor. In one embodiment, the inhibitor of checkpoint is an inhibitory antibody (e.g., a monospecific antibody such as a monoclonal antibody). The antibody may be, e.g., humanized or fully human. In some embodiments, the inhibitor of checkpoint is a fusion protein, e.g., an Fc-receptor fusion protein. In some embodiments, the inhibitor of checkpoint is an agent, such as an antibody, that interacts with a checkpoint protein. In some embodiments, the inhibitor of checkpoint is an agent, such as an antibody, that interacts with the ligand of a checkpoint protein. In some embodiments, the inhibitor of checkpoint is an inhibitor (e.g., an inhibitory antibody or small molecule inhibitor) of CTLA-4 (e.g., an anti-CTLA4 antibody such as ipilimumab/Yervoy or tremelimumab). In some embodiments, the inhibitor of checkpoint is an inhibitor (e.g., an inhibitory antibody or small molecule inhibitor) of PD-1 (e.g., nivolumab/Opdivo®; pembrolizumab/Keytruda®; pidilizumab/CT-011). In some embodiments, the inhibitor of checkpoint is an inhibitor (e.g., an inhibitory antibody or small molecule inhibitor) of PDL1 (e.g., MPDL3280A/RG7446; MED14736; MSB0010718C; BMS 936559). In some embodiments, the inhibitor of checkpoint is an inhibitor (e.g., an inhibitory antibody or Fc fusion or small molecule inhibitor) of PDL2 (e.g., a PDL2/Ig fusion protein such as AMP 224). In some embodiments, the inhibitor of checkpoint is an inhibitor (e.g., an inhibitory antibody or small molecule inhibitor) of B7-H3 (e.g., MGA271), B7-H4, BTLA, HVEM, TIM3, GAL9, LAG3, VISTA, KIR, 2B4, CD160, CGEN-15049, CHK1, CHK2, A2aR, B-7 family ligands, or a combination thereof.

In some embodiments, the compound of the invention is used in combination with another anti-cancer therapy used for the treatment of uveal melanoma such as surgery, a MEK inhibitor, and/or a PKC inhibitor, or a combination thereof. For example, in some embodiments, the method further comprises performing surgery prior to, subsequent to, or at the same time as administration of the compound of the invention. In some embodiments, the method further comprises administration of a MEK inhibitor (e.g., selumetinib, binimetinib, or tametinib) and/or a PKC inhibitor (e.g., sotrastaurin or IDE196) prior to, subsequent to, or at the same time as administration of the compound of the invention.

In any of the combination embodiments described herein, the first and second therapeutic agents are administered simultaneously or sequentially, in either order. The first therapeutic agent may be administered immediately, up to 1 hour, up to 2 hours, up to 3 hours, up to 4 hours, up to 5 hours, up to 6 hours, up to 7 hours, up to, 8 hours, up to 9 hours, up to 10 hours, up to 11 hours, up to 12 hours, up to 13 hours, 14 hours, up to hours 16, up to 17 hours, up 18 hours, up to 19 hours up to 20 hours, up to 21 hours, up to 22 hours, up to 23 hours up to 24 hours or up to 1-7, 1-14, 1-21 or 1-30 days before or after the second therapeutic agent.

### Pharmaceutical Compositions

A crystalline form described herein (e.g., the crystalline forms of the compound of formula (I)) may be formulated into pharmaceutical compositions for administration to human subjects in a biologically compatible form suitable for administration in vivo. Pharmaceutical compositions typically include an active agent as described herein and a physiologically acceptable excipient (e.g., a pharmaceutically acceptable excipient). Formulation principles for the compound of formula (I) have been described in WO 2020/160180, the disclosure of which is incorporated by reference herein in its entirety. The crystalline forms described herein are especially beneficial for solid pharmaceutical compositions, e.g., solid dosage forms (e.g., tablets, powders, lozenges, sachets, cachets, and soft and hard gelatin capsules).

The compound of formula (I) of the invention may be administered, for example, by oral, parenteral, buccal, sublingual, nasal, rectal, patch, pump, or transdermal administration and the pharmaceutical compositions formulated accordingly. Parenteral administration includes intravenous, intraperitoneal, subcutaneous, intramuscular, transepithelial, nasal, intrapulmonary, intrathecal, rectal, and topical modes of administration. Parenteral administration may be by continuous infusion over a selected period of time. Preferably, a crystalline form of the compound of formula (I) is administered orally.

Suitable pharmaceutical carriers, as well as pharmaceutical necessities for use in pharmaceutical formulations, are described in Remington: The Science and Practice of Pharmacy, 21^{st} Ed., Gennaro, Ed., Lippencott Williams & Wilkins (2005), a well-known reference text in this field, and in the USP/NF (United States Pharmacopeia and the National Formulary).

### Unit Dosage Forms

A crystalline form described herein (e.g., the second crystalline form of the compound of formula (I)) may be formulated into a unit dosage form for oral administration (e.g., a capsule). The crystalline form of the compound of formula (I) (e.g., the second crystalline form of the compound of formula (I)) may be supplied in different capsule strengths for oral administration (e.g., 1 to 2.5 mg, 2.5 to 5 mg, 5 to 10 mg, 10 to 15 mg, 15 to 20 mg, 20 to 25 mg, or 50 to 100 mg). In some examples, the crystalline form of the compound of formula (I) is supplied in 2.5 mg or 20 mg capsule strengths for oral administration.

The unit dosage form may contain suitable pharmaceutical carriers and excipients as described in the pharmaceutical compositions section. Excipients may include, for example: antiadherents, antioxidants, binders, coatings, compression aids, disintegrants, dyes (colors), emollients, emulsifiers, fillers (diluents), film formers or coatings, flavors, fragrances, glidants (flow enhancers), lubricants, preservatives, printing inks, sorbents, suspending or dispersing agents, sweeteners, and waters of hydration. The unit dosage form may contain one or more of a filler, a disintegrant, a wetting agent, a glidant, a lubricant, and a capsule shell.

In some examples, the filler may be 70 to 90% (w/w) of the unit dosage form. The filler may be microcrystalline cellulose, mannitol, or a combination thereof.

In some examples, the disintegrant may be 4 to 6% (w/w) of the unit dosage form. The disintegrant may be croscarmellose sodium.

In some examples, the wetting agent may be 0.5 to 1.5% (w/w) of the unit dosage form. The wetting agent may be sodium auryl sulfate.

In some examples, the glidant may be 1.5 to 2.5% (w/w) of the unit dosage form. The glidant may be colloidal silicon dioxide.

In some examples, the lubricant may be 0.4 to 0.6% (w/w) of the unit dosage form. The lubricant may be magnesium stearate.

In some examples, the capsule shell is made of a polymeric shell. The polymeric shell can be made from hypromellose and titanium dioxide.

A crystalline form described herein (e.g., the second crystalline form of the compound of formula (I)) may be formulated into a unit dosage form for oral administration (e.g., a capsule) as described in Table 1.

TABLE 1

| | | 2.5 mg Capsule | | 20 mg Capsule | |
|---|---|---|---|---|---|
| | | Amount | | Amount | |
| | | per | | per | |
| | | Capsule | Content | Capsule | Content |
| Component | Function | (mg) | (%) | (mg) | (%) |
| Second crystalline | Active | 2.50 | 2.63 | 20.00 | 17.39 |
| form of a compound | ingredient | | | | |
| of formula (I) | | | | | |
| Microcrystalline | Filler | 48.24 | 50.78 | 48.20 | 41.91 |
| cellulose | | | | | |
| Mannitol | Filler | 36.18 | 38.08 | 36.95 | 32.13 |
| Croscarmellose | Disintegrant | 4.75 | 5.00 | 5.80 | 5.04 |
| Sodium | | | | | |
| Sodium lauryl sulfate | Wetting | 0.95 | 1.00 | 1.15 | 1.00 |
| | agent | | | | |
| Colloidal silicon | Glidant | 1.90 | 2.00 | 2.30 | 2.00 |
| dioxide | | | | | |
| Magnesium stearate | Lubricant | 0.48 | 0.51 | 0.60 | 0.52 |
| Size 4, Swedish | Capsule | 1 capsule | — | — | — |
| Orange HPMC | Shell | | | | |
| Capsules | | | | | |
| Size 3, Blue Green | Capsule | — | — | 1 capsule | — |
| HPMC Capsules | Shell | | | | |
| Total | | 95.00 | 100 | 115.00 | 100 |

The composition of the Swedish orange hypromellose capsule shells is described in Table 2.

TABLE 2

| Composition of | | |
|---|---|---|
| capsule body and | | |
| cap | Function | Content |
| FDA/E172 RED | Colorant | 1.1817% |
| IRON OXIDE | | |
| TITANIUM DIOXIDE | Opacifier | 0.4916% |
| Hypromellose | Structure | qs 100% |

The composition of blue green hypromellose capsule shells is described in Table 3.

TABLE 3

| Composition of | | |
|---|---|---|
| capsule body and | | |
| cap | Function | Content |
| FD&C Blue #1 | Colorant | 0.0281% |
| FD&C Yellow #5 | Colorant | 0.0069% |
| TITANIUM DIOXIDE | Opacifier | 2.2306% |
| Hypromellose | Structure | qs 100% |

### EXAMPLES

The abbreviations below are used throughout the examples section
2-MeTHF 2-Methyl tetrahydrofuran
BSA Benzenesulfonic acid
EtOAc Ethyl acetate
EtOH Ethanol
iPrOAc Isopropyl acetate
iPrOH Isopropanol
KOH Potassium hydroxide
MeCN Acetonitrile
MEK Methyl ethyl ketone
MeOH Methanol
MgSO₄ Magnesium sulfate
MTBE Methyl tert-butyl ether
NaOH Sodium hydroxide
THF Tetrahydrofuran
TSA p-Toluenesulfonic acid

### Example 1. Preparation of the Crystalline Forms of the Compound of Formula (I)

Preparation of the compound of formula (I) is described in WO 2020/160180, the disclosure of which is incorporated by reference herein in its entirety.

### The First Crystalline Form of the Compound of Formula (I).

A reactor with overhead stirring was charged with the compound of formula (I) (250.0 g) and acetonitrile (1250 mL). The contents were stirred at 22° C. for 5 minutes until all solid had dissolved. Purified water (125 mL) and seed (50 mg) were then charged and the mixture was stirred for 10 minutes. Purified water (125 mL) and seed (50 mg) were then charged and the mixture was stirred for 30 minutes. Purified water (250 mL) and seed (100 mg) were then charged and the mixture was stirred for 1 hour. Purified water (875 L) was charged intermittently over the next 21 hours and the reaction was slowly cooled to 9° C. The slurry was filtered and washed with purified water (500 mL). The damp solid was dried under vacuum at 40° C. with a nitrogen purge for 19 hours. Product was obtained as a beige solid (180.3 g). The dried compound of formula (I) (180.3 g) was charged to a 5 L jacketed reactor with overhead stirring and MTBE (901 mL) was added. The slurry was stirred at 7° C. for 1 hour. The slurry was filtered and washed with MTBE (200 mL, 7° C.). The damp solid was dried under vacuum at 40° C. with a nitrogen purge for 24 hours. Pure compound of formula (I) was obtained as an off-white powder (161.2 g), having an XRPD diffractogram consistent with the first crystalline form of the of the compound of formula (I).

### The Second Crystalline Form of the Compound of Formula (I).

A 5 L jacketed reactor with overhead stirring was charged with the compound of formula (I) (349.1 g) and acetonitrile (2250 mL, 6.5 vols with respect to the compound of formula (I) weight). The contents were heated to 75° C. and aged for 15 minutes to dissolve all the solid. The brown solution was cooled to 28° C. over 30 minutes and seeded (250 mg) to give a very light seed bed. Purified water (2.3 L) was charged over the next 4 hours. The slurry was cooled to 4° C. The slurry was then stirred for 14 hours. The slurry was filtered and washed with a mixture of acetonitrile (250 mL) and purified water (500 mL), pre-cooled to 5° C. The damp solid was dried under vacuum at 40° C. with a nitrogen purge for 30 hours. The product was obtained as an off-white solid (278.78 g).

A 5 L jacketed reactor with overhead stirring was charged with the compound of formula (I) (700.8 g, obtained from the previous step) and MTBE (3000 mL). The contents were stirred at 20° C. for 15 minutes and then cooled to 5° C. and stirred for 60 minutes. The slurry was filtered and washed with MTBE (1000 mL, pre-cooled to 5° C.). The damp solid was dried under vacuum at 40° C. with a nitrogen purge for 18 hours. Pure compound of formula (I) was obtained as an off-white powder (691.6 g), having an XRPD diffractogram consistent with the second crystalline form of the of the compound of formula (I).

### Example 2. Analysis of the Crystalline Forms of the Compound of Formula (I)

### X-Ray Powder Diffraction.

X-Ray Powder Diffraction patterns were collected on a Bruker D2 Phaser Gen 2 using Cu Kα radiation (30 kV, 10 mA), θ-θ goniometer, divergence slit (0.2 mm) and an SSD160 (1 D Mode) Detector with a 4.7990 opening. The software used for data collection was Diffrac.Commander version 6.5.0.1 and the data was presented using Diffra.Eva version 4.2.1.11.XRPD diffractograms were acquired under ambient conditions via reflection on a flat silica zero background plate with rotation at 15 revolutions per minute. The data collection range was 3.0-4(%) 2 with a step size of 0.020250 and a collection time of 0.25 seconds per step. Samples were prepared by placing powder onto a flat silica zero background plate and ensuring the sample surface is flat.

The XRPD peak list for the first crystalline form of the compound of formula (I) is provided in Table 4, and the XRPD peak list for the second crystalline form of the compound of formula (I) is provided in Table 5.

TABLE 4

| | | | | | Relative |
|---|---|---|---|---|---|
| | Angle | | Net | Gross | Intensity |
| No. | (°2θ) | d Value (Å) | Intensity | Intensity | (%) |
| 1 | 9.862 | 8.96179 | 40.8 | 66.4 | 6.2 |
| 2 | 10.269 | 8.60739 | 79.9 | 105.0 | 12.1 |
| 3 | 10.521 | 8.40146 | 95.5 | 120.0 | 14.5 |
| 4 | 10.821 | 8.16932 | 48.1 | 70.8 | 7.3 |
| 5 | 11.650 | 7.58961 | 92.3 | 116.0 | 14.0 |
| 6 | 11.829 | 7.47514 | 178.0 | 202.0 | 27.1 |
| 7 | 12.251 | 7.21876 | 104.0 | 128.0 | 15.8 |
| 8 | 14.375 | 6.15661 | 167.0 | 192.0 | 25.4 |
| 9 | 14.903 | 5.93985 | 312.0 | 339.0 | 47.3 |
| 10 | 15.190 | 5.82815 | 282.0 | 310.0 | 42.8 |
| 11 | 16.023 | 5.52702 | 184.0 | 212.0 | 28.0 |
| 12 | 16.295 | 5.43544 | 151.0 | 178.0 | 22.9 |
| 13 | 17.369 | 5.10142 | 659.0 | 686.0 | 100.0 |
| 14 | 18.452 | 4.80441 | 226.0 | 254.0 | 34.3 |
| 15 | 19.065 | 4.65142 | 471.0 | 497.0 | 71.4 |
| 16 | 19.358 | 4.58160 | 53.1 | 79.1 | 8.1 |
| 17 | 20.589 | 4.31029 | 332.0 | 361.0 | 50.3 |
| 18 | 21.199 | 4.18773 | 548.0 | 577.0 | 83.2 |
| 19 | 23.190 | 3.83247 | 134.0 | 161.0 | 20.4 |
| 20 | 24.121 | 3.68657 | 23.6 | 47.2 | 3.6 |
| 21 | 24.266 | 3.66499 | 107.0 | 130.0 | 16.3 |
| 22 | 25.161 | 3.53653 | 230.0 | 251.0 | 34.9 |
| 23 | 26.298 | 3.38614 | 62.8 | 81.9 | 9.5 |
| 24 | 26.635 | 3.34411 | 144.0 | 163.0 | 21.9 |
| 25 | 28.225 | 3.15924 | 93.8 | 110.0 | 14.2 |
| 26 | 28.653 | 3.11303 | 10.1 | 26.1 | 1.5 |
| 27 | 31.082 | 2.87505 | 24.0 | 37.5 | 3.6 |
| 28 | 31.660 | 2.82381 | 15.4 | 28.8 | 2.3 |
| 29 | 35.498 | 2.52681 | 23.1 | 39.8 | 3.5 |
| 30 | 36.400 | 2.46625 | 28.8 | 44.9 | 4.4 |
| 31 | 39.541 | 2.27730 | 26.0 | 39.6 | 3.9 |

TABLE 5

| | | | | | Relativ |
|---|---|---|---|---|---|
| | Angle | d Value | Net | Gross | Intensity |
| No. | (°2θ) | (Å) | Intensity | Intensity | (%) |
| 1 | 7.655 | 11.53919 | 943.0 | 968.0 | 70.4 |
| 2 | 8.341 | 10.59243 | 261.0 | 288.0 | 19.5 |
| 3 | 9.369 | 9.43180 | 1340.0 | 1366.0 | 100.0 |
| 4 | 10.643 | 8.30546 | 170.0 | 190.0 | 12.7 |
| 5 | 12.080 | 7.32059 | 138.0 | 153.0 | 10.3 |
| 6 | 13.665 | 6.47491 | 22.7 | 37.4 | 1.7 |
| 7 | 14.822 | 5.97215 | 986.0 | 1005.0 | 73.6 |
| 8 | 15.288 | 5.79109 | 37.8 | 58.7 | 2.8 |
| 9 | 15.892 | 5.57208 | 546.0 | 567.0 | 40.7 |
| 10 | 16.996 | 5.21276 | 1323.0 | 1344.0 | 98.7 |
| 11 | 17.657 | 5.01891 | 22.4 | 43.5 | 1.7 |
| 12 | 18.757 | 4.72715 | 547.0 | 573.0 | 40.9 |
| 13 | 19.227 | 4.61258 | 42.5 | 69.0 | 3.2 |
| 14 | 19.979 | 4.44064 | 536.0 | 564.0 | 40.0 |
| 15 | 20.554 | 4.31770 | 163.0 | 190.0 | 12.2 |
| 16 | 21.239 | 4.17989 | 92.4 | 116.0 | 6.9 |
| 17 | 22.012 | 4.03493 | 161.0 | 185.0 | 12.1 |
| 18 | 22.759 | 3.90404 | 92.1 | 116.0 | 6.9 |
| 19 | 23.026 | 3.85936 | 223.0 | 247.0 | 16.6 |
| 20 | 23.722 | 3.74769 | 274.0 | 300.0 | 20.4 |
| 21 | 24.322 | 3.65655 | 132.0 | 158.0 | 9.9 |
| 22 | 25.241 | 3.52552 | 151.0 | 174.0 | 11.3 |
| 23 | 26.017 | 3.42209 | 182.0 | 204.0 | 13.6 |
| 24 | 28.174 | 3.16483 | 19.3 | 38.0 | 1.4 |
| 25 | 29.079 | 3.06831 | 156.0 | 174.0 | 11.6 |
| 26 | 30.833 | 2.89770 | 42.4 | 57.2 | 3.2 |
| 27 | 31.943 | 2.79946 | 12.6 | 25.4 | 0.9 |
| 28 | 32.735 | 2.73356 | 21.3 | 34.2 | 1.6 |
| 29 | 33.722 | 2.65577 | 18.6 | 31.8 | 1.4 |
| 30 | 34.750 | 2.57952 | 63.5 | 77.9 | 4.7 |
| 31 | 35.682 | 2.51422 | 26.9 | 41.7 | 2.0 |
| 32 | 36.108 | 2.48552 | 35.8 | 51.0 | 2.7 |
| 33 | 36.712 | 2.44602 | 20.2 | 36.3 | 1.5 |
| 34 | 37.971 | 2.36775 | 36.3 | 51.1 | 2.7 |
| 35 | 38.616 | 2.32969 | 14.2 | 28.5 | 1.1 |

### Differential Scanning Calorimetry (DSC) and Thermal Gravimetric Analysis (TGA).

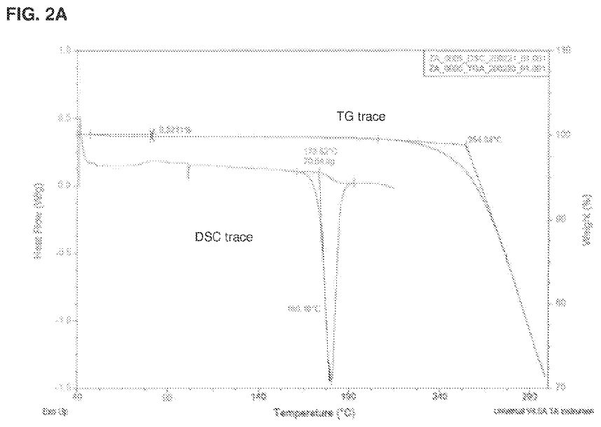

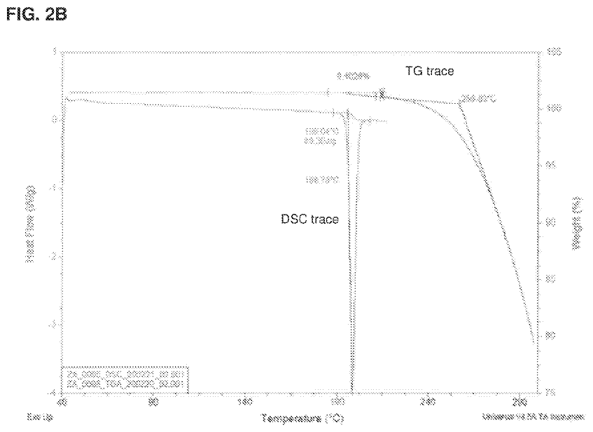

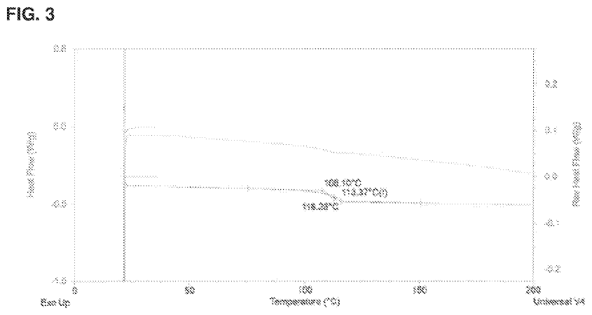

DSC data was collected on a TA Instruments Q2000 DSC. A predefined amount of the sample, 2.0 to 10.0 mg, was placed in an aluminum pan and heated at 5° C./minute from 25° C. to 220° C. A purge of dry nitrogen at 100 mL/minute was maintained over the sample. The instrument control and data acquisition are acquired using Q Advantage software release version 5.5.23. The data was processed and presented using the TA Universal Analysis 2000 software version 4.5A build 4.5.0.5. FIG. 2A shows the DSC trace for the first crystalline form of the compound of formula (I) and FIG. 2B shows the DSC trace for the second crystalline form of the compound of formula (I). FIG. 3 shows the modulated DSC performed for the first crystalline form of the compound of formula (I) with two heating cycles.

TGA data was collected on a TA Instruments Q5000 TGA. A predefined amount of the sample, 2.0 to 10.0 mg, was placed in an aluminum pan and heated at 10° C./minute from 40° C. to 300° C., or varied as experimentation dictated. A purge of dry nitrogen at 25 mL/minute was maintained over the sample. The instrument control and data acquisition are acquired using Q Advantage software release version 5.5.23. The data was processed and presented using the TA Universal Analysis 2000 software version 4.5A build 4.5.0.5. FIG. 2A shows the TGA trace for the first crystalline form of the compound of formula (I) and FIG. 2B shows the TGA trace for the second crystalline form of the compound of formula (I).

The First Crystalline Form of the Compound of Formula (I).

TGA and DSC analysis exhibited low temperature weight loss of 0.3% (w/w) due to loss of the free water in the sample, and molecular degradation from 220° C. with onset at 254.3° C. (FIG. 2A). A melting endotherm was observed at 180.1° C. with an onset at 173.5° C. Modulated DSC performed with two heating cycles showed a glass transition temperature between 108.1° C. and 116.3° C. during the second cycle, indicating that under these measurement conditions the material remained amorphous after melting during the first heating cycle (FIG. 3).

The Second Crystalline Form of the Compound of Formula (I).

TGA and DSC analysis exhibited molecular degradation at 240° C. with no low temperature weight loss (FIG. 2B). A melting endotherm was observed at 198.8° C. with an onset at 196.0° C., accompanied by a weight loss of 0.4% (w/w), indicating the inclusion of solvent in the crystal structure that is only released upon melting. Modulated DSC performed with two heating cycles showed a glass transition temperature between 108.1° C. and 116.3° C. during the second cycle, indicating that under these measurement conditions the material remained amorphous after melting during the first heating cycle (FIG. 3).

### Dynamic Vapor Sorption (DVS).

Dynamic vapor sorption (DVS) was carried out using the TA Instruments Q5000 SA. A predefined amount of the sample, 2.0 to 10.0 mg, was placed in a platinum pan. The sample was allowed to equilibrate at 50° C. at 0% RH for a period of 60 minutes. The sample was then equilibrated at 25° C. before ramping the humidity from 0 to 95% RH at 5% increments every hour. A similar ramp profile was used for desorption cycle. XRPD analysis was also performed on post DVS sample.

The First Crystalline Form of the Compound of Formula (I).

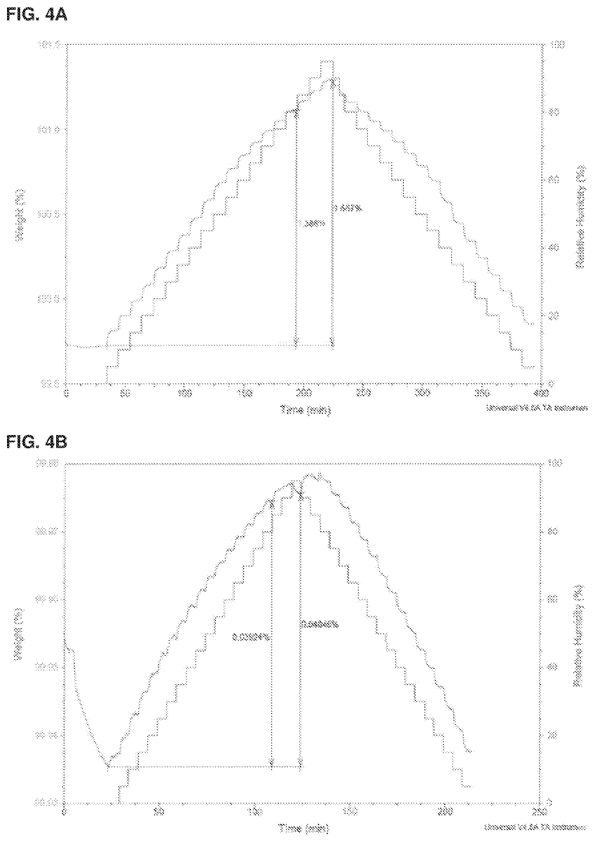

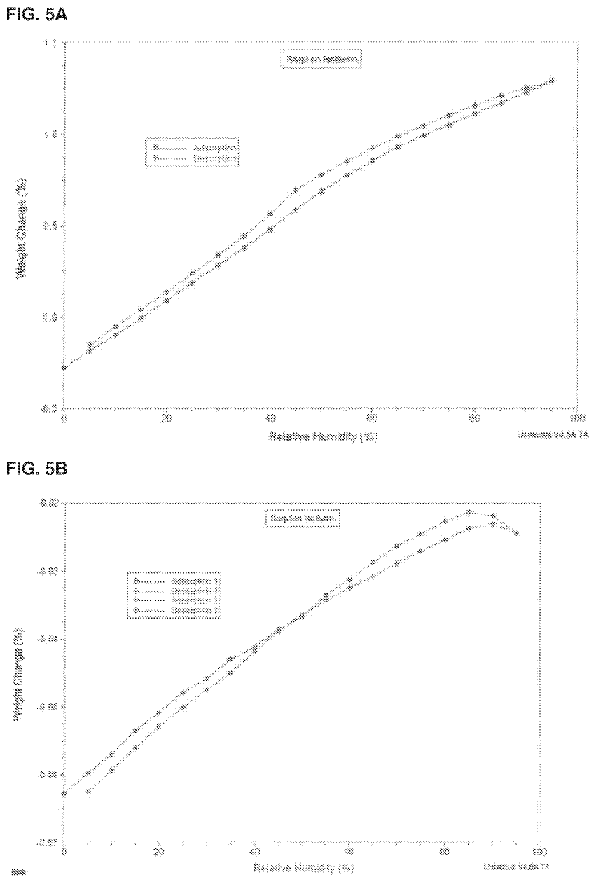

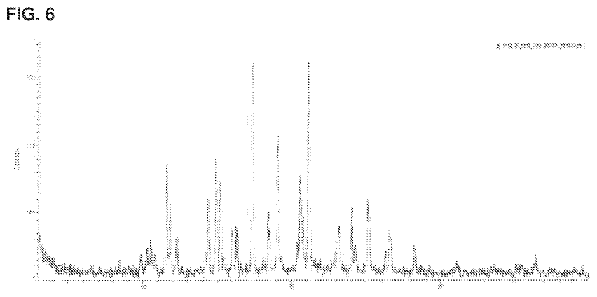

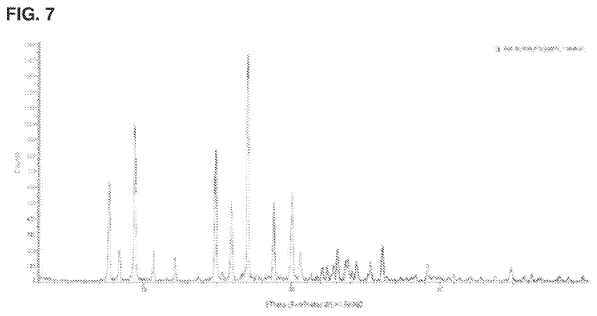

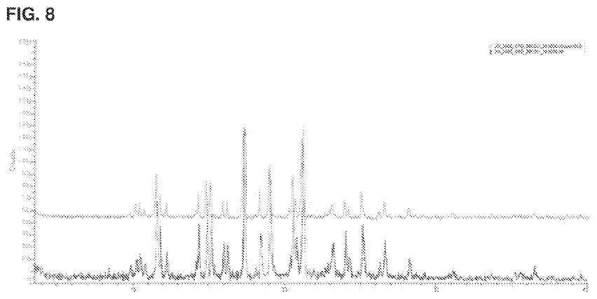

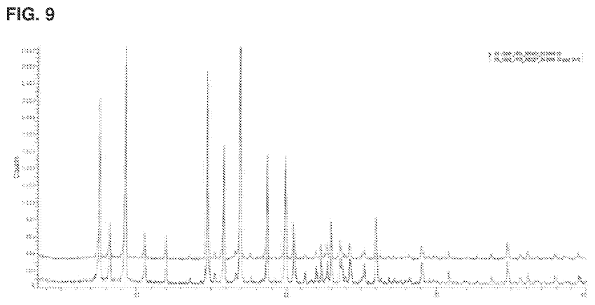

DVS analysis showed a weight increase of 1.4% (w/w) during exposure to 80% RH, hence the material is classified as slightly hygroscopic (FIG. 4A). Slight hysteresis is observed but XRPD analysis performed on the sample after DVS showed that no structural changes occurred as a result of exposure to high humidity.

The Second Crystalline Form of the Compound of Formula (I).

DVS analysis showed an increase of 0.04% (w/w) during exposure to water up to 80% RH, indicating that the material is non-hygroscopic (FIG. 4B). XRPD analysis performed on the sample after DVS showed that no structural changes occurred as a result of exposure to high humidity.

### Nuclear Magnetic Resonance (NMR).

¹H NMR was performed by dissolving the samples at approximately 10 mg/mL in 0.6 mL volume of deuterated DMSO-d6 and referenced to the TMS at 0.00 ppm before analyzing by Bruker 400 MHz NMR Spectrometer using the parameters shown in Table 6 below. The sample was analyzed using Top Spin software.

TABLE 6

| Method Name | Parameters | Value |
|---|---|---|
| Proton.icon (identity | Pulse angle | 30° |
| determination) | Spectral width (SW) | 30 ppm |
| | Transmitter offset | 5 ppm |
| | (O1P) | |
| | Digitazation (TD) | 131 K |
| | Number of scans (NS) | ≥32 |
| | Pulse repetition time | ≥3 s |
| | (D1) | |

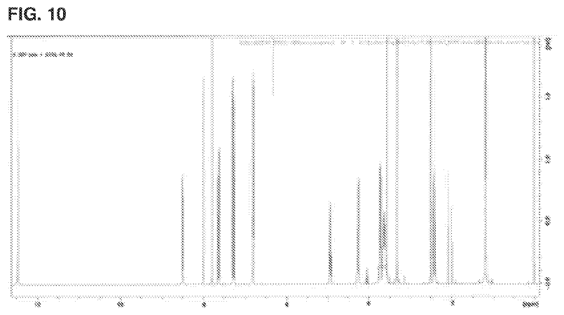

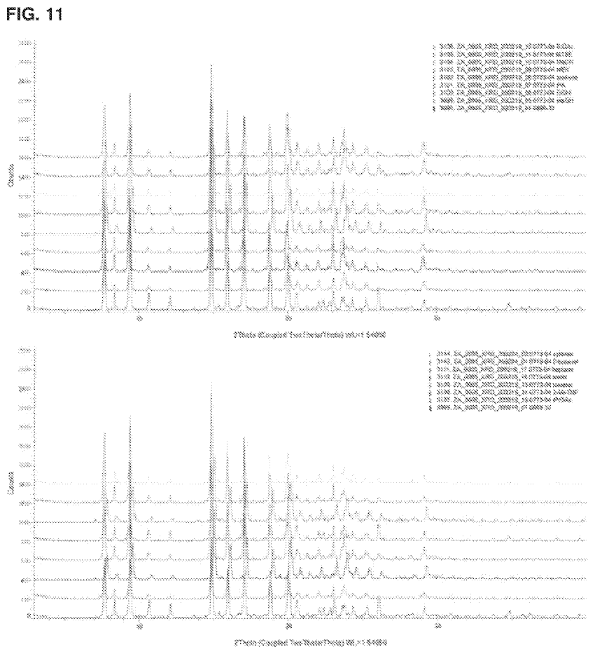

FIG. 10 shows the ¹H NMR spectrum of the second crystalline form of the compound of formula (I).

### Polarized Light Microscopy (PLM).

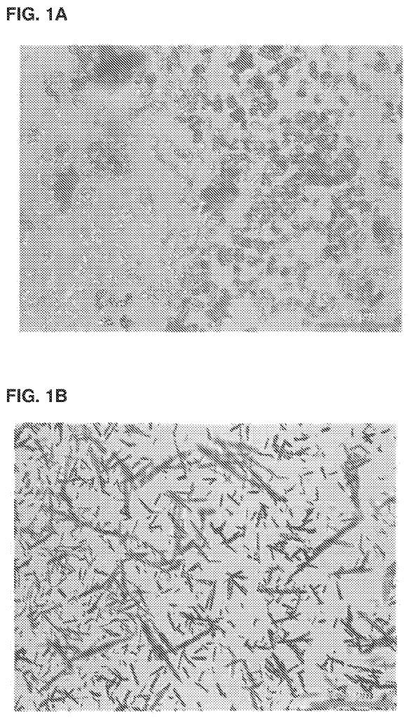

Optical microscopy was performed using the Nikon Eclipse E400 Optical Microscope equipped with 2, 4, 10, 25, and 40× objective lens. The microscope is coupled with a QImaging digital camera. The camera is controlled by the proprietary QCapture Pro 7 software. The camera software measurement tool is calibrated using a calibration slide (Pyser-SGI S8 Micrometer scale 1 mm/0.01 mm). Samples were prepared either by distributing a powder over a microscope slide with mineral oil or by placing a drop of suspension directly onto a microscope slide. A cover slip was then placed on top. FIG. 1A shows a microscopic image of the first crystalline form of the compound of formula (I) and FIG. 1B shows a microscopic image of the second crystalline form of the compound of formula (I).

The First Crystalline Form of the Compound of Formula (I).

Optical microscopy showed the material consists mostly of fine particles less than 20 μm in length (FIG. 1A).

The Second Crystalline Form of the Compound of Formula (I).

Optical microscopy showed the material consists mostly of columnar crystals of up to 250 μm in length (FIG. 1B).

### Example 3. Screening for Stable Forms Via Slurry Conversions

### Single Solvent Slurry Screen.

A single solvent slurry screen was conducted to allow phase transformations of the second crystalline form of the compound of formula (I) to a more thermodynamically stable form in single solvent systems (Table 7). Saturated solutions were prepared with additional crystalline compound (e.g., the second crystalline form of the compound of formula (I)) in quantities sufficient to perform solid state analysis on resulting samples. Magnetic stirrers were added to vials, and the suspensions were allowed to stir at room temperature for 4 days. Solid samples generated were analyzed via XRPD to assess crystallinity and crystal structure.

TABLE 7

| Solvent | Crystal Structure |
|---|---|
| Methanol | Second crystalline form of the compound of formula (I) |
| Ethanol | Second crystalline form of the compound of formula (I) |
| Isopropanol | Second crystalline form of the compound of formula (I) |
| 2-Butanol | Second crystalline form of the compound of formula (I) |
| Acetone | Second crystalline form of the compound of formula (I) |
| MEK | Second crystalline form of the compound of formula (I) |
| Acetonitrile | Second crystalline form of the compound of formula (I) |
| MTBE | Second crystalline form of the compound of formula (I) |
| Ethyl acetate | Second crystalline form of the compound of formula (I) |
| Isopropyl | Second crystalline form of the compound of formula (I) |
| acetate | |
| 2-MeTHF | Second crystalline form of the compound of formula (I) |
| THF | Dissolved |
| Toluene | Second crystalline form of the compound of formula (I) |
| Xylenes | Second crystalline form of the compound of formula (I) |
| Water | Second crystalline form of the compound of formula (I) |
| n-Heptane | Second crystalline form of the compound of formula (I) |

The second crystalline form of the compound of formula (I) could not be slurried in THF as the solubility is extremely high and the API dissolved. Solid samples were obtained from slurries in all other solvents screened. XRPD analysis showed that each solid sample exhibits the same diffraction pattern, and hence the same crystal structure, as the second form crystalline form of the compound of formula (I) (FIG. 11). Each diffraction pattern displays the characteristic reflections of the second crystalline form of the compound of formula (I), with reflections at 7.5 °2θ, 9.5 °2θ, 15.0 °2θ, and 17.0 °2θ.

### Temperature Cycled Single Solvent Slurry Screen.

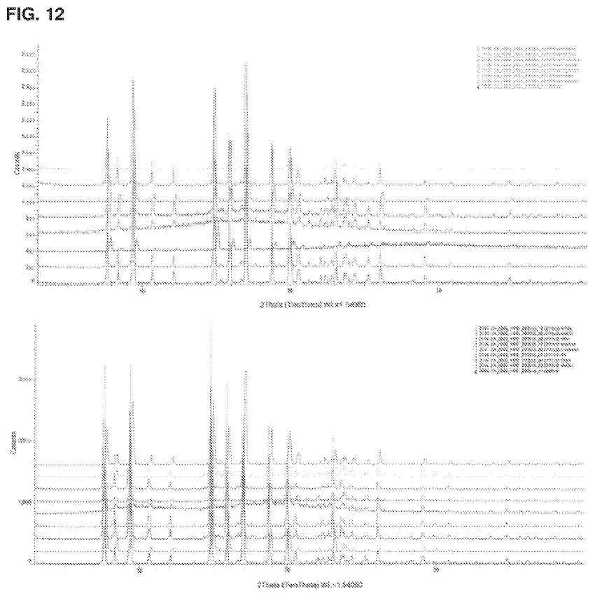

A single solvent slurry screen was conducted to allow phase transformations of the second crystalline form of the compound of formula (I) to a more thermodynamically stable form in single solvent systems (Table 8). Saturated solutions were prepared with additional crystalline compound (e.g., the second crystalline form of the compound of formula (I)) in quantities sufficient to perform solid state analysis on resulting samples. Magnetic stirrers were added to vials, and the suspensions were cycled between 50° C. and 5° C. for three cycles with agitation. Solid samples generated were analyzed via XRPD to assess crystallinity and crystal structure. The second crystalline form of the compound of formula (I) could not be slurried in THE as the solubility is extremely high and the API dissolved. Solid samples were obtained from slurries in all other solvents screened. XRPD analysis showed that each solid sample exhibits the same diffraction pattern, and hence the same crystal structure, as the second form crystalline form of the compound of formula (I) (FIG. 12). Each diffraction pattern displays the characteristic reflections of the second crystalline form of the compound of formula (I), with reflections at 7.5 °2θ, 9.5 °2θ, 15.0 °2θ, and 17.0 °2θ, although samples obtained from 2-butanol and xylenes exhibited poor crystallinity.

TABLE 8

| Solvent | Crystal Structure |
|---|---|
| Methanol | Second crystalline form of the compound of formula (I) |
| Ethanol | Second crystalline form of the compound of formula (I) |
| Isopropanol | Second crystalline form of the compound of formula (I) |
| 2-Butanol | Second crystalline form of the compound of formula (I) |
| | (low crystallinity) |
| Acetone | Second crystalline form of the compound of formula (I) |
| MEK | Second crystalline form of the compound of formula (I) |
| Acetonitrile | Second crystalline form of the compound of formula (I) |
| MTBE | Second crystalline form of the compound of formula (I) |
| Ethyl acetate | Second crystalline form of the compound of formula (I) |
| Isopropyl | Second crystalline form of the compound of formula (I) |
| acetate | |
| 2-MeTHF | Second crystalline form of the compound of formula (I) |
| THF | Dissolved |
| Toluene | Second crystalline form of the compound of formula (I) |
| Xylenes | Second crystalline form of the compound of formula (I) |
| | (low crystallinity) |
| Water | Second crystalline form of the compound of formula (I) |
| n-Heptane | Second crystalline form of the compound of formula (I) |

### Binary Solvent Slurry Screen.

A binary solvent slurry screen was conducted to allow phase transformations of the second crystalline form of the compound of formula (I) to a more thermodynamically stable form in binary solvent systems (Table 9). Solvent combinations were selected based on solvent properties, functionality, and miscibility. Saturated solutions were prepared in 50/50 (v/v) solvent mixtures with additional crystalline compound (e.g., the second crystalline form of the compound of formula (I)) in quantities sufficient to perform solid state analysis on resulting samples. Magnetic stirrers were added to vials, and the suspensions were allowed to stir at room temperature for 5 days.

TABLE 9

| Solvent 1 | Solvent 2 | Crystal Structure |
|---|---|---|
| Methanol | Toluene | Second crystalline form of the compound of |
| | | formula (I) |
| Methanol | THF | Dissolved |
| Methanol | Acetonitrile | Second crystalline form of the compound of |
| | | formula (I) |
| Methanol | Ethyl | Second crystalline form of the compound of |
| | acetate | formula (I) |
| Toluene | THF | Second crystalline form of the compound of |
| | | formula (I) |
| Toluene | Acetonitrile | Second crystalline form of the compound of |
| | | formula (I) |
| Toluene | Ethyl | Second crystalline form of the compound of |
| | acetate | formula (I) |
| n-Heptane | THF | Second crystalline form of the compound of |
| | | formula (I) |
| n-Heptane | Ethyl | Second crystalline form of the compound of |
| | acetate | formula (I) |
| THF | Acetonitrile | Second crystalline form of the compound of |
| | | formula (I) |
| THF | Ethyl | Second crystalline form of the compound of |
| | acetate | formula (I) (low crystallinity) |
| Acetonitrile | Ethyl | Second crystalline form of the compound of |
| | acetate | formula (I) |

Solid samples were obtained from slurries in all solvent mixtures screened except methanol/THF as the solubility was high. XRPD analysis showed that each solid sample exhibits the same diffraction pattern, and hence the same crystal structure, as the second crystalline form of the compound of formula (I). Each diffraction pattern displays the characteristic reflections of the second crystalline form of the compound of formula (I), with reflections at 7.5 °2θ, 9.5 °2θ, 15.0 °2θ, and 17.0 °2θ, although samples obtained from THF/ethyl acetate exhibited poor crystallinity.

### Example 4. Screening for Metastable Forms Via Nucleation

Excess compound of formula (I) (e.g., the second crystal form of the compound of formula (I)) was slurried in each solvent at 45° C. for 2 hours before separating the solids and liquors by centrifugation. The liquors were transferred to fresh vials for low temperature evaporation, high temperature evaporation and cooling crystallization screen.

### Low Temperature Evaporation Screen.

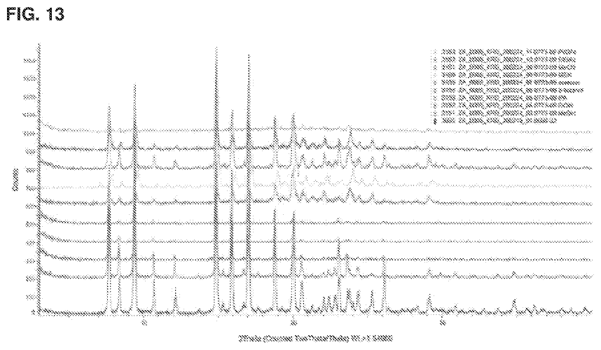

A low temperature evaporation screen was conducted to isolate crystal structures that may form as a result of super-saturation generated by slow solvent evaporation under ambient conditions (Table 10). Sufficient material for analysis was not obtained from toluene, xylenes, water, and n-heptane. The evaporation of THF and 2-MeTHF resulted in an oil, and the second crystalline form of the compound of formula (I) was formed in all other solvents (FIG. 13).

TABLE 10

| Solvent | Crystal Structure |
|---|---|
| Methanol | Second crystalline form of the compound of formula (I) |
| Ethanol | Second crystalline form of the compound of formula (I) |
| Isopropanol | Second crystalline form of the compound of formula (I) |
| 2-Butanol | Second crystalline form of the compound of formula (I) |
| Acetone | Second crystalline form of the compound of formula (I) |
| MEK | Second crystalline form of the compound of formula (I) |
| Acetonitrile | Second crystalline form of the compound of formula (I) |
| MTBE | Second crystalline form of the compound of formula (I) |
| Ethyl acetate | Second crystalline form of the compound of formula (I) |
| Isopropyl | Second crystalline form of the compound of formula (I) |
| acetate | |
| 2-MeTHF | Oil |
| THF | Oil |
| Toluene | N/A |
| Xylenes | N/A |
| Water | N/A |
| n-Heptane | N/A |

### High Temperature Evaporation Screen.

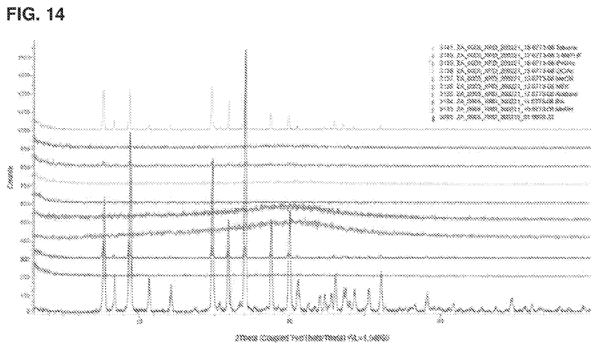

A high temperature evaporation screen was conducted to isolate metastable phases that may form as a result of super-saturation generated by rapid solvent evaporation (Table 11). The liquors were heated from 45° C. to 50° C. to ensure all solids were fully dissolved before the solvent was evaporated at 50° C. A controlled air flow across the vials of −0.8 bar was generated using the Crystal Breeder. Once evaporation was complete, solid samples were analyzed using XRPD to assess crystallinity and crystal structure (FIG. 14).

TABLE 11

| Solvent | Crystal Structure |
|---|---|
| Methanol | Amorphous |
| Ethanol | N/A |
| Isopropanol | Second crystalline form of the compound of formula (I) |
| 2-Butanol | N/A |
| Acetone | Amorphous |
| MEK | Amorphous |
| Acetonitrile | Amorphous |
| MTBE | N/A |
| Ethyl acetate | Amorphous |
| Isopropyl | Second crystalline form of the compound of formula (I) |
| acetate | |
| 2-MeTHF | Oil |
| THF | Oil |
| Toluene | Second crystalline form of the compound of formula (I) |
| Xylenes | N/A |
| Water | N/A |
| n-Heptane | N/A |

The compound of formula (I) converted to an oil phase after the evaporation of 2-MeTHF and THF. Sufficient material for analysis was not obtained from ethanol, 2-butanol, MTBE, xylenes, water, and n-heptane. Solid samples were obtained from the evaporation of all other solvents screen. XRPD analysis showed that material produced by the evaporation of isopropanol, isopropyl acetate, and toluene formed the second crystalline form of the compound of formula (I). All other solid samples were amorphous.

### Cooling Crystallization Screen.

A cooling crystallization screen was conducted to isolate metastable phases that may form as a result of super-saturation generated by rapid decrease in temperature (Table 12). The liquors were heated to 50° C. to ensure all solids were fully dissolved. The vials were then cooled to 5° C. at 20° C./minute and held at low temperature overnight. Any solid samples generated were isolated and analyzed via XRPD to assess crystallinity and crystal structure.

TABLE 12

| Solvent | Crystal Structure |
|---|---|
| Methanol | Second crystalline form of the compound of formula (I) |
| Ethanol | Second crystalline form of the compound of formula (I) |
| Isopropanol | Second crystalline form of the compound of formula (I) |
| 2-Butanol | N/A |
| Acetone | N/A |
| MEK | Second crystalline form of the compound of formula (I) |
| Acetonitrile | Second crystalline form of the compound of formula (I) |
| MTBE | N/A |
| Ethyl acetate | Second crystalline form of the compound of formula (I) |
| Isopropyl | N/A |
| acetate | |
| 2-MeTHF | N/A |
| THF | N/A |
| Toluene | N/A |
| Xylenes | N/A |
| Water | N/A |
| n-Heptane | N/A |

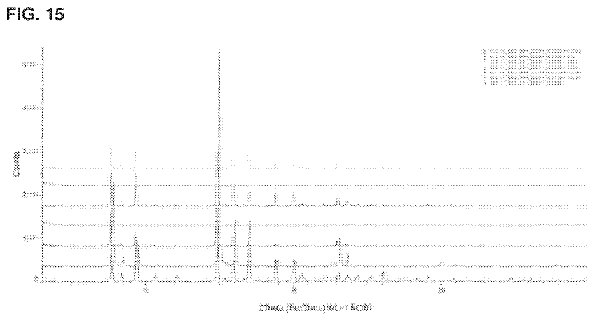

Small quantities of solid samples were obtained by crash cooling in methanol, ethanol, isopropanol, MEK, acetonitrile, and ethyl acetate. XRPD analysis showed that each solid sample is consistent with the second crystalline form of the compound of formula (I) (FIG. 15). Crystallinity appeared low in samples from ethanol and isopropanol; however, this could be due to small sample size. In all other solvents screened, the compound of formula (I) remained in solution, indicating the maximum concentration generated in this study does not approach the meta-stable zone boundary at 5° C.

### Hydrate Screen.

A hydrate screen was conducted to observe solvated or hydrated structures that may be formed from the compound of formula (I) in solvent/water mixtures (Table 13). Solvents were selected based on miscibility with water. 0.5 mL aliquots of solvent were added to approximately 50 mg of the second crystalline form of the compound of formula (I) until the solids were dissolved in the minimum solvent required. 1 mL aliquots of water were added until a cloud point was observed or until 20 mL had been added. The vials were heated to 50° C. and cooled to room temperature. After stirring at room temperature for 2 days, the solvent was allowed to evaporate to increase the material available for characterization. When sufficient crystals had formed, a sample was removed for initial XRPD analysis. The remaining solvent was evaporated until dry to produce a larger sample for further characterization, with the crystals already present acting as a seed.

TABLE 13

| Solvent/ | Solvent:water | Crystal | Crystal |
|---|---|---|---|
| water | ratio | Structure Wet | Structure Dry |
| Methanol | 1:1 | Second crystalline | Second crystalline |
| | | form + reflections | form |
| Ethanol | 1:1 | Second crystalline | Second crystalline |
| | | form | form |
| Isopropanol | 3:2 | Second crystalline | Second crystalline |
| | | form | form |
| Acetonitrile | 1:2 | Second crystalline | Second crystalline |
| | | form | form |
| Acetone | 1:1 | Second crystalline | Second crystalline |
| | | form | form |
| THF | 1:2 | Amorphous | Amorphous |

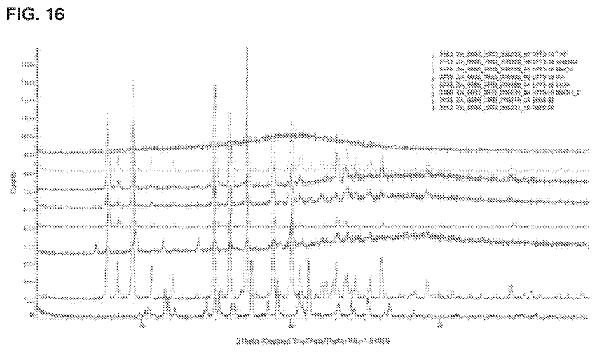

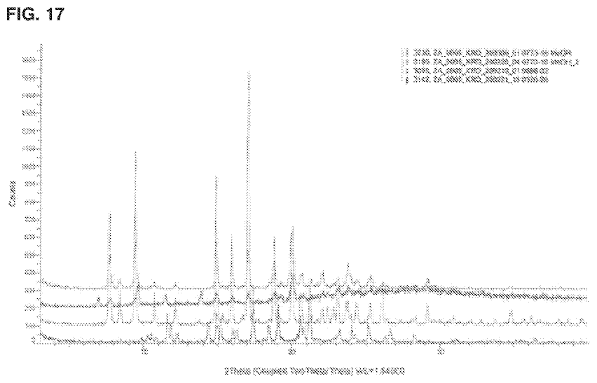

Initial analysis was performed on wet samples to ensure any alterations resulting from drying were observed. XRPD showed that the sample obtained from THF/water was amorphous and all other samples have the second crystalline form of the compound of formula (I) (FIG. 16). However, the sample obtained from methanol/water exhibits additional reflections at 11.5 °2θ (which may correspond to the first crystalline form of the compound of formula (I)) and 7 °2θ (which was not assigned). XRPD analysis on the dry sample showed only the second crystalline form of the compound of formula (I) remains (FIG. 17).

### Example 5. Competitive Suspension Equilibration Study

A competitive suspension equilibration study was carried out to identify the relative stability of the two crystalline forms of the compound of formula (I) in aqueous and organic solvent systems. Competitive suspension equilibration experiments were conducted in ethyl acetate, 50% methanol/water (v/v) and 50% acetonitrile/water (v/v) at room temperature and 60° C. The second crystalline form of the compound of formula (I) was added to each solvent to create a saturated solution. Further crystalline forms of the compound of formula (I) (e.g., the first and second crystalline forms of the compound of formula (I)) were added for conversion material and to act as seeds. Each suspension was allowed to stir for 7 or 14 days, and were analyzed via XRPD to determine which form the solids had converted to. Table 14 summarizes the competitive suspension equilibration conditions.

TABLE 14

| Solvent | Temperature | Crystal Structure |
|---|---|---|
| Ethyl acetate | room temperature | Second crystalline form of the |
| | | compound of formula (I) |
| Ethyl acetate | 60° C. | Second crystalline form of the |
| | | compound of formula (I) |
| Methanol | room temperature | Second crystalline form of the |
| | | compound of formula (I) |
| Methanol | 60° C. | Second crystalline form of the |
| | | compound of formula (I) |
| Acetonitrile | room temperature | Second crystalline form of the |
| | | compound of formula (I) |
| Acetonitrile | 60° C. | Second crystalline form of the |
| | | compound of formula (I) |
| Methanol/water* | room temperature | First + second crystalline forms |
| | | of the compound of formula (I) |
| Methanol/water* | 60° C. | First + second crystalline forms |
| | | of the compound of formula (I) |
| Acetonitrile/water | room temperature | First + second crystalline forms |
| | | of the compound of formula (I) |
| Acetonitrile/water | 60° C. | First + second crystalline forms |
| | | of the compound of formula (I) |

*Suspension stirred for 14 days

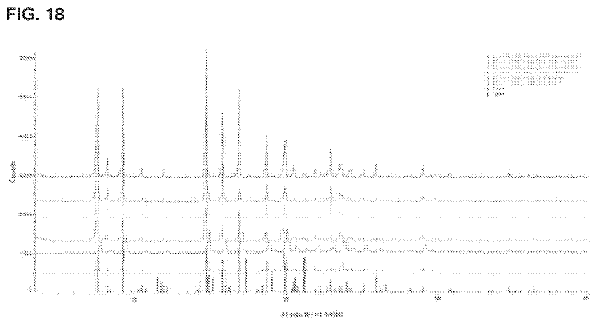

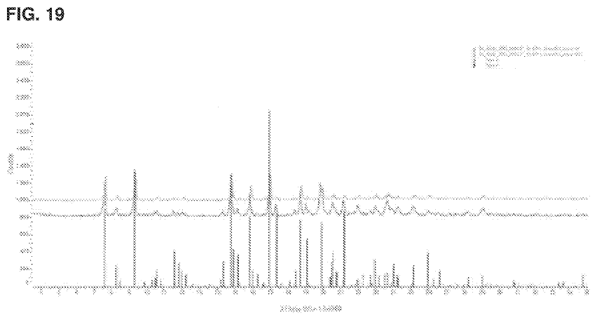

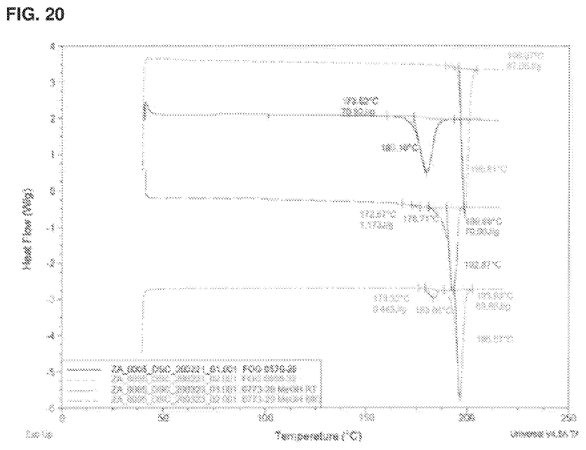

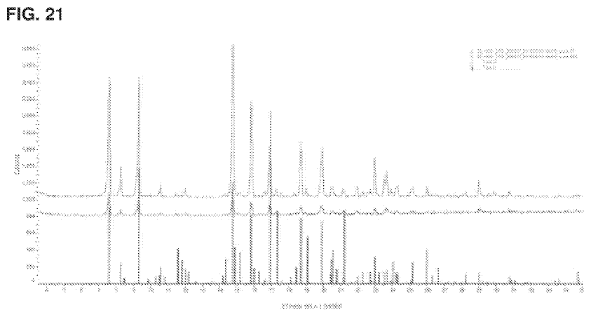

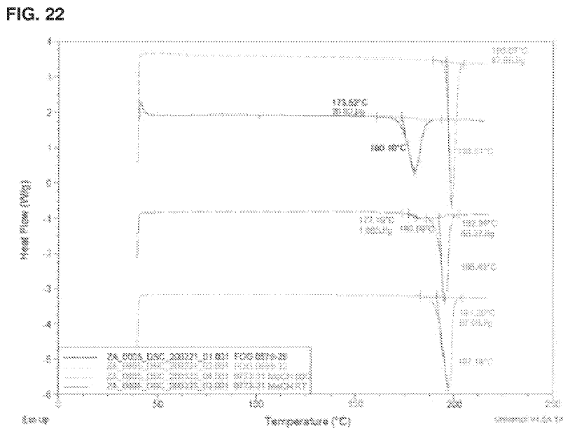

XRPD analysis showed that the compound of formula (I) fully converted to the second crystalline form within seven days of stirring both crystalline forms at room temperature and 60° C. in ethyl acetate, methanol, and acetonitrile (FIG. 18). However, stirring in aqueous solvents did not result in the conversion of either crystalline form to the other. XRPD analysis of solids stirred for 14 days in methanol/water showed a majority of the second crystalline form, with reflections characteristic of the first crystalline form visible at 14.4 °2θ and 17.4 °2θ (FIG. 19). Although it appears from this analysis that the second crystalline form is the dominant form, this may be due to a greater proportion of the second crystalline form in the input material. DSC analysis also showed a mixture of the first and second crystalline forms, although there is a shift in the melting point for each form due to the impurity presence of the competing structures (FIG. 20). Similarly, XRPD analysis of solids stirred for seven days in acetonitrile/water showed the second crystalline form of the compound of formula (I) with additional reflections at 14.4 °2θ and 17.4 °2θ, corresponding to the first crystalline form of the compound of formula (I) (FIG. 21). DSC analysis showed the first and second crystalline forms of the compound of formula (I) are present when stirred at room temperature; however, only the first crystalline form was detected when stirred at 60° C. (FIG. 22).

### Example 6. Salt and Co-Crystal Screen

A salt screen of the compound of formula (I) was carried out with a variety of counterions to identify crystalline salt and co-crystal forms that may be used for purification purposes or to influence the solubility and stability of the final solid form.

The potential for salt and co-crystal crystallization was evaluated via liquid assisted grinding, a slow evaporation screen, slurrying, and temperature cycling of the compound of formula (I) combined with 15 counterions, and in a selection of 16 solvents (Table 15). This allowed the investigation to survey a broad array of conditions that may influence the formation and stability of crystalline salt and co-crystal forms.

TABLE 15

| Solvents | Counterions |
|---|---|
| Methanol | Benzenesulfonic acid (BSA) |
| Ethanol/water | p-toluenesulfonic acid (TSA) |
| Isopropanol | Hydrochloric acid (HCl) |
| 2-Butanol | Sulfuric acid |
| Acetone | Malonic acid |
| Acetonitrile | Oxalic acid |
| Ethyl acetate | Phosphoric acid |
| Isopropyl acetate | Ethane disulfonic acid (EdSA) |
| Toluene | Serine |
| Xylenes | Lysine |
| THF | Glutamic acid |
| MTBE | Magnesium sulfate |
| | Sodium hydroxide |
| | Potassium hydroxide |

### Liquid Assisted Grinding Screen.

Counterions were screened via liquid assisted grinding to encourage the formation of co-crystals by minimizing the influence of solvent-molecule interactions. Approximately 50 mg of the second crystalline form of the compound of formula (I) was combined with 50 mg counterion in a mortar and pestle (Table 15). Two to five drops of methanol were added and the mixture was ground in periods of 2 minutes, followed by analysis by XRPD. Grinding was repeated as necessary.

All mixtures with acidic counterions formed a thick gel that showed mixtures of the input components of the second crystalline form of the formula of compound 1 and the respective acids once solidified. Mixtures with basic counterions remained as free flowing powders; however, the composition did not alter from the input components of the second crystalline form and the respective counterions.

### Evaporative Screen.

A stock solution of the compound of formula (I) (0.1 M) was made in acetone, and 150 μL was dispensed into each well across two 96 well plates. 0.1 M stock solutions of each acid were made in methanol or water and dispensed at an equimolar ratio into 12 wells each. The solvent was allowed to evaporate until nitrogen flow, after which 250 μL of each crystallization solvent was added to the API/counterion mixtures and agitated to ensure mixing. The arrangement was such that, each row on the well plate represented a counterion and each column represented a crystallization solvent used for the salt screen. The wells were covered with parafilm with a pin hole, and the solvents were allowed to evaporate slowly at room temperature.

All powder samples obtained from the screen were analyzed via XRPD to identify crystalline salts. All other samples were first studied by PLM and where birefringence was observed, XRPD was also performed. Crystalline samples were grouped by their crystal structure and named by the counterion.

Many crystalline samples were identified as the second crystalline form of the compound of formula (I) or a mixture of the second crystalline form and the input counterion. Novel reflections were observed when the compound of formula (I) was combined with serine, magnesium sulfate, sodium hydroxide, and potassium hydroxide (Table 16). Novel crystal structures identified by XRPD were analyzed by DSC, and where possible by ¹H NMR.

TABLE 16

| | | EtOH/ | | 2- | | | | | |
|---|---|---|---|---|---|---|---|---|---|
| | MeOH | water | iPrOH | Butanol | MeCN | EtOAc | iPrOAc | Xylenes | MTBE |
| Serine | | | Serine | | | | | | Serine |
| | | | + | | | | | | + |
| | | | U | | | | | | U |
| MgSO₄ | Type | MG1 | MG1 | MG1 | Type | Type | Type | MG1 | MG1 |
| | B + | | | | B + | B + | B + | | |
| | MG1 | | | | MG1 | MG1 | MG1 | | |
| NaOH | | D | D | | D | D | D | D | D |
| KOH | | | | | | D | | | |

U = unidentified reflections;
D = degradant;
Type B = the second crystalline form of the compound of formula (I);
MG1 = magnesium salt.

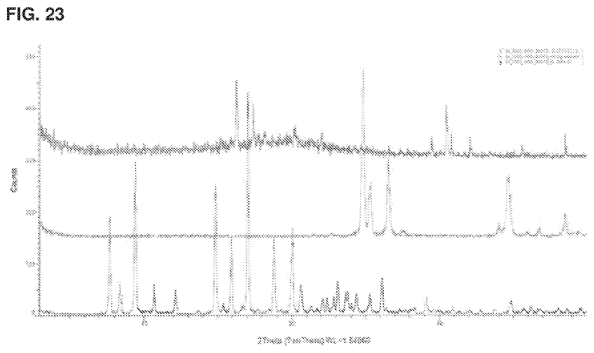

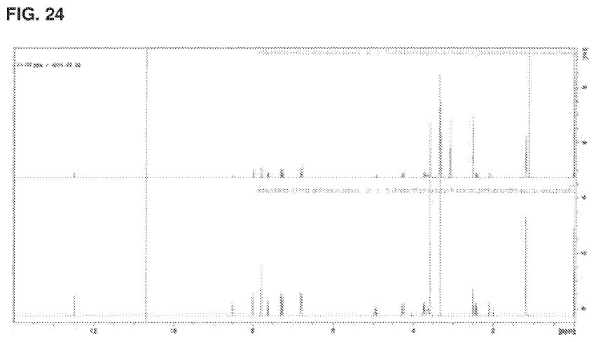

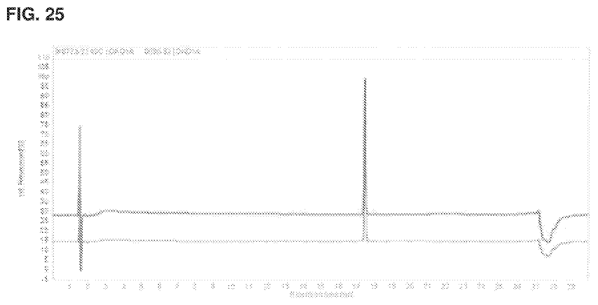

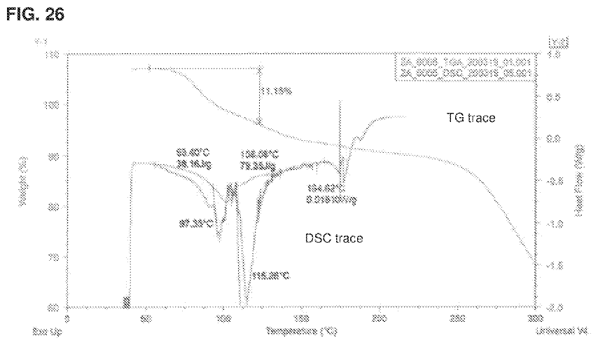

The XRPD analysis of solids formed by the combination of the compound of formula (I) with magnesium sulfate showed a new diffraction pattern, and hence, a new crystal structure (FIG. 23). No molecular degradation was visible via ¹H NMR spectroscopy and HPLC analysis, indicating successful co-crystal formation, identified as MG1 (FIG. 24, FIG. 25). TG and DSC analysis showed significant low temperature weight loss of 11% (w/w) with a corresponding low temperature endotherm suggesting solvent evaporation (FIG. 26). A melting endotherm was observed at 115° C., followed by degradation from 165° C.

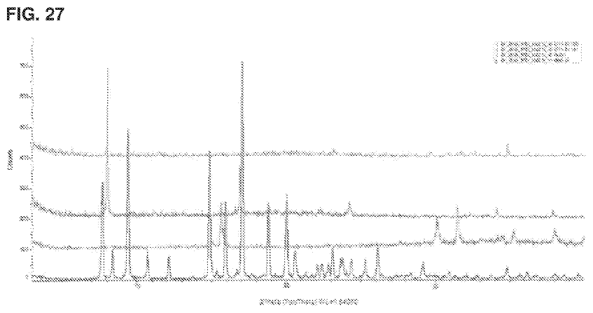

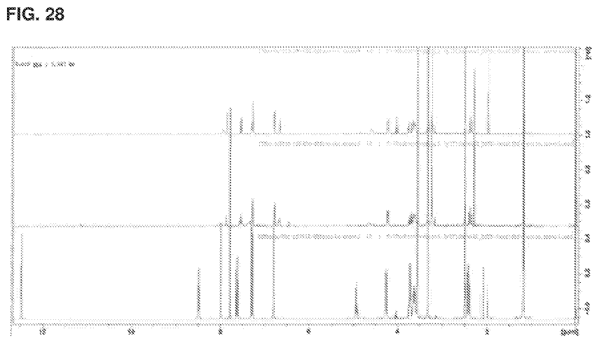

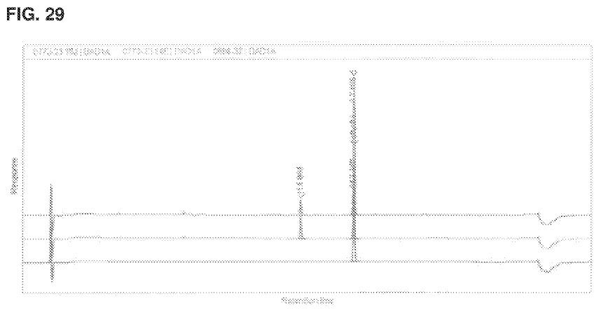

The XRPD analysis of solids formed by the combination of the compound of formula (I) with sodium hydroxide and potassium hydroxide showed a new diffraction pattern, and hence, a new crystal structure (FIG. 27). However, molecular degradation was visible via ¹H NMR spectroscopy in the increased noise of the baseline and HPLC analysis showed a new molecule eluting at 14.7 minutes (FIG. 28, FIG. 29). This suggests the new structure is a product of molecular degradation in a basic environment.

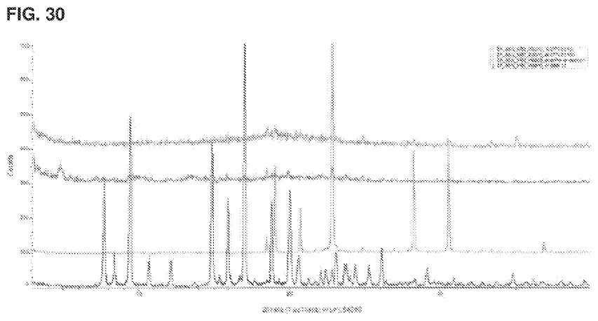

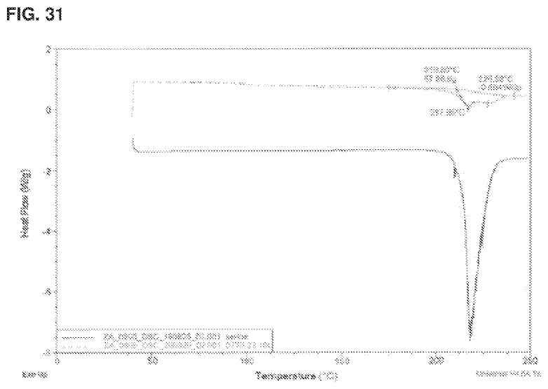

The XRPD analysis of solids formed by the combination of the compound of formula (I) with serine in isopropanol and MTBE showed previously unobserved reflections at low intensity, suggesting the early stages of formation of a new structure (FIG. 30). DSC analysis showed a melting endotherm at 217° C. which corresponds to serine melting temperature, followed by a second endotherm at 227° C. which potentially indicates the presence of a second structure (FIG. 31). Further characterization was not performed due to insufficient material, so serine was combined with the compound of formula (I) in isopropanol and MTBE and temperature cycled between 20° C. and 50° C. (for 10 cycles) in an attempt to recreate the new structure. However, the reproduction of the novel diffraction pattern was unsuccessful as XRPD analysis showed the samples are mixtures of serine and the second crystalline form of the compound of formula (I).

Amorphous slurries. Amorphous samples formed in the evaporative screen were transferred to vials, and the appropriate solvents were added dropwise to form a suspension. The suspensions were allowed to stir at room temperature for 2 days to allow transformation to crystalline structures. The solvent was then evaporated at room temperature, allowing any potential crystals present to seed further crystallization, in order to extract sufficient material for analysis.

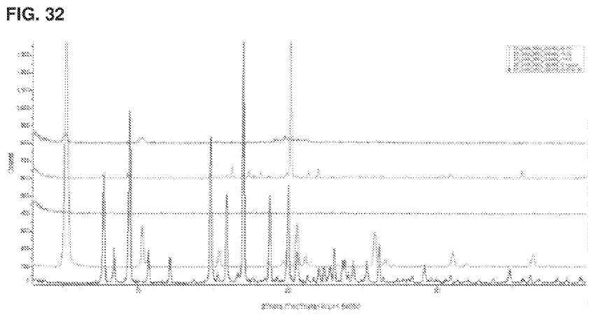

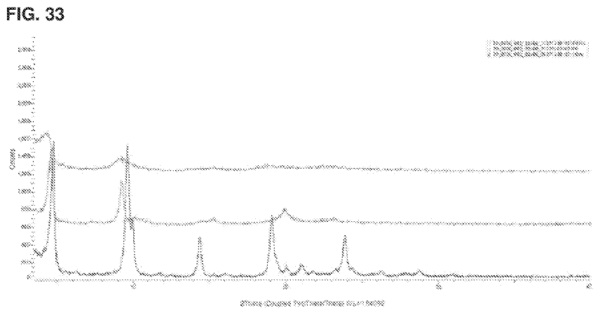

Solid samples were obtained with HCl in ethyl acetate, magnesium sulfate in toluene, and lysine in xylenes. XRPD analysis showed the HCl material was amorphous, the magnesium sulfate material showed reflections characteristic of the second crystal form of the compound of formula (I) only, and the lysine material showed lysine reflections (FIG. 32). All other slurried samples formed oils.

### Temperature Cycling in Toluene.

Based on the results of the evaporative screen, which showed a high percentage of solid amorphous material formed in toluene, the second crystalline form of the compound of formula (I) was suspended with 9 acids and temperature cycled between 5° C. and 50° C. (for 10 cycles) to encourage salt crystallization (Table 17). This experiment was also performed with HCl, oxalic acid, and ethane disulfonic acid in ethyl acetate. Solid samples were analyzed by XRPD, which showed that no new crystal structures were formed.

TABLE 17

| Acid | Solvent | Outcome |
|---|---|---|
| BSA | Methanol | Gel |
| TSA | Ethanol/water | Gel |
| Sulphuric acid | Isopropanol | Viscous solution |
| Malonic acid | 2-Butanol | The second crystalline form of the |
| | | compound of formula (I) |
| Maleic acid | Acetone | The second crystalline form of the |
| | | compound of formula (I) |
| Oxalic acid | Acetonitrile | The second crystalline form of the |
| | | compound of formula (I) |
| Phosphoric | Ethyl acetate | Oil |
| acid | | |
| HCl | Isopropyl | Gel |
| | acetate | |
| EdSA | Toluene | Gel |
| HCl | Xylenes | Viscous solution |
| Oxalic acid | THF | The second crystalline form of the |
| | | compound of formula (I) |
| EdSA | MTBE | Gel |

### Reactive Screen in Ethyl Acetate.

The second crystalline form of the compound of formula (I) was combined with 8 acids in 1:1 equivalents, and ethyl acetate was added until dissolved or up to a maximum of 50 mL. Heptane was then added until a cloud point was observed. Amorphous material was initially generated but quickly converted to gels on the vial walls. The vials were allowed to stir at room temperature for up to 10 days and solid samples were analyzed by XRPD. Solids were formed when the compound of formula (I) was combined with oxalic acid and malonic acid; however, XRPD data showed the isolated solids were amorphous with oxalic acid, and were the second crystalline form of the compound of formula (I) with malonic acid. Table 18 summarizes the outcomes of the reactive screen of the compound of formula (I) with acidic counterions in ethyl acetate/heptane.

TABLE 18

| Acid | Outcome |
|---|---|
| Malonic acid | The second crystalline form of the |
| | compound of formula (I) |
| Maleic acid | Gel |
| Oxalic acid | Amorphous |
| TSA | Gel |
| BSA | Gel |
| HCl, sulfuric acid, | Did not dissolve |
| EdSA | |

Vials from the reactive screen were temperature cycled between 20° C. and 50° C. (for 10 cycles) to encourage crystallization. Solid material was observed in combinations with malonic acid, HCl, and ethane disulfonic acid; however, XRPD analysis showed the second crystalline form of the compound of formula (I) or amorphous diffraction patterns.

### Hydrochloride Salt.

Several experiments were carried out in ethyl acetate to generate the hydrochloride salt:
The second crystalline form of the compound of formula (I) was suspended with HCl in ethyl acetate (1:1 equivalents) and allowed to stir for 3 days.
The second crystalline form of the compound of formula (I) was fully dissolved in ethyl acetate with HCl (1:1 equivalents), and 33% heptane anti-solvent was added to initiate nucleation. The vial was then temperature cycled between 20° C. and 50° C. (10 cycles) to encourage crystallization.
The second crystalline form of the compound of formula (I) was fully dissolved in ethyl acetate with HCl (1:1 equivalents) and a small amount of HCl salt seed was added to the solution before the addition of heptane. The suspension was allowed to stir at 30° C. for 3 days.

XRPD analysis showed that the unseeded experiments yield amorphous solids. However, the seeded sample exhibited low intensity reflections corresponding to the hydrochloride salt (HCl1).

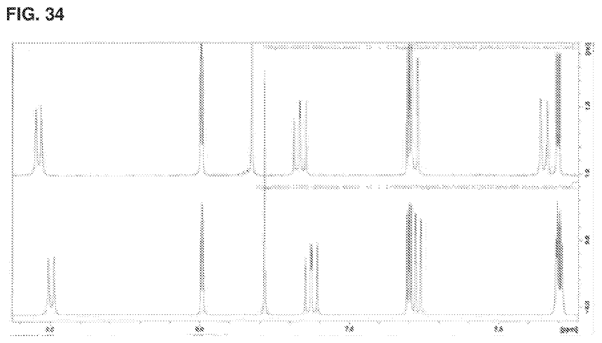

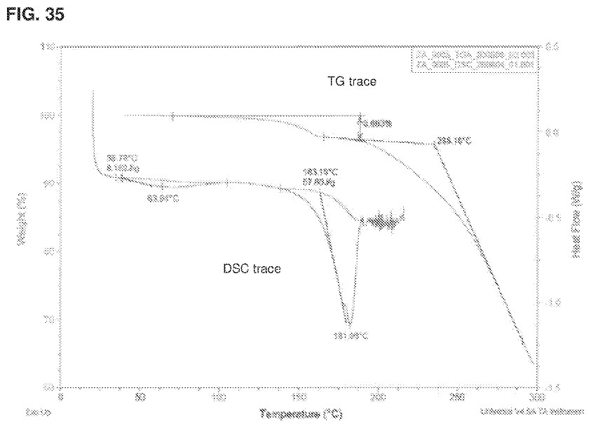

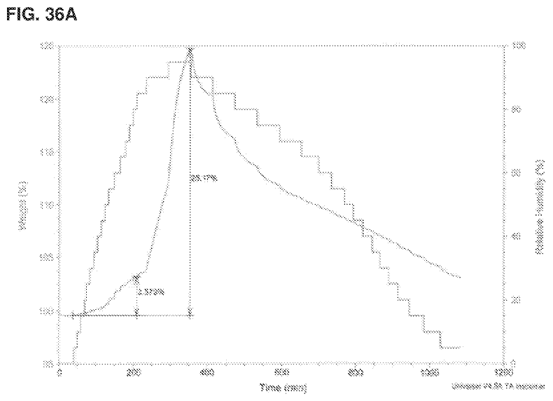

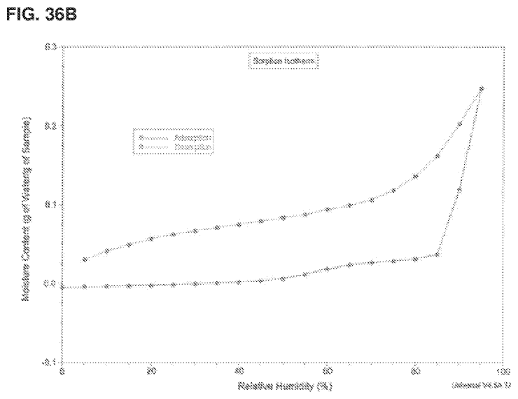

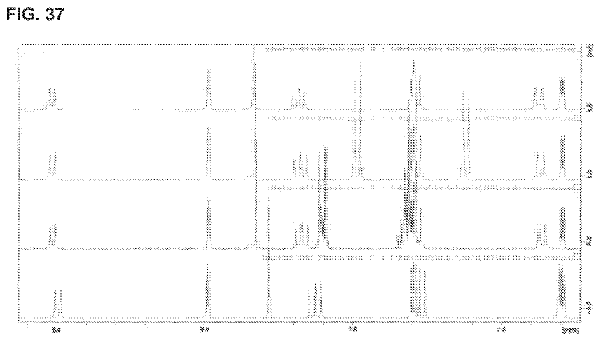

A reactive screen using anhydrous concentrated HCl was performed in ethyl acetate, isopropyl acetate and isopropanol. The second crystalline form of the compound of formula (I) was suspended in ethyl acetate and HCl was added in 1:1 equivalents, which rapidly initiated nucleation. XRPD analysis showed the hydrochloride salt was successfully formed in all three solvents, with greatest crystallinity in ethyl acetate (FIG. 33). ¹H NMR analysis confirmed the formation of a salt without degradation (FIG. 34). TGA and DSC data of the sample formed in ethyl acetate exhibited DSC data of the sample formed in ethyl acetate exhibited weight loss of 3.7% (w/w) at approximately 150° C., and molecular degradation from 195° C. with onset at 235° C. (FIG. 35). A melting endotherm was observed at 182° C. with an onset at 163.2° C., followed by degradation. DVS analysis showed a weight increase of 3.6% (w/w) during exposure to 80% RH, hence the material is classified as hygroscopic, with a total weight increase of 25.2% (w/w) during exposure to 95% RH (FIG. 36). After data collection, the sample was found to have deliquesced.

### Reactive Screen in Isopropyl Acetate.

The second crystalline form of the compound of formula (I) was combined with 5 acids in 1:1 equivalents, and isopropyl acetate was added until dissolved or up to a maximum of 60 mL. Heptane was then added until a cloud point was observed. XRPD analysis showed that all solids formed in isopropyl acetate were amorphous. However, ¹H NMR spectroscopy on the solids showed a shift in resonance corresponding to protons on the aromatic group with no indication of degradation, suggesting that salts had been formed (FIG. 37). TGA analysis indicates the ethane disulfonic acid salt is highly unstable, whereas degradation occurs between 150° C. and 180° C. for the other salts. Modulated DSC performed with two heating cycles was performed on the salts. The besylate showed a glass transition temperature between 97.7° C. and 112.1° C., and the tosylate showed a glass transition temperature between 94.7° C. and 107.2° C. during the second cycle, indicating that under these measurement conditions the materials remained amorphous after melting. ¹H NMR analysis was not carried out on the potential oxalate salt as it was insoluble in DMSO, hence it could not be confirmed as a salt. Table 19 summarizes the reactive salt screen of the compound of formula (I) with acidic counterions in isopropyl acetate/heptane.

TABLE 19

| Acid | Outcome |
|---|---|
| BSA | Amorphous |
| | salt |
| TSA | Amorphous |
| | salt |
| EdSA | Amorphous |
| | salt |
| Sulphuric | Gum |
| acid | |
| Oxalic acid | Amorphous |
| | solid |

### OTHER EMBODIMENTS

While the invention has been described in connection with specific embodiments thereof, it will be understood that invention is capable of further modifications and this application is intended to cover any variations, uses, or adaptations of the invention following, in general, the principles of the invention and including such departures from the present disclosure that come within known or customary practice within the art to which the invention pertains and may be applied to the essential features hereinbefore set forth, and follows in the scope of the claims.

Other embodiments are in the claims.

1. A crystalline form of the compound of formula (I): 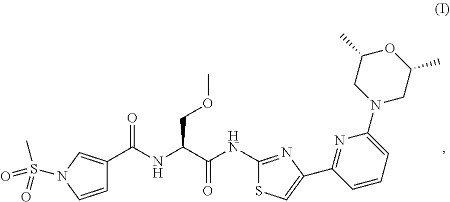 [SMILES: COC[C@H](NC(=O)c1ccn(S(C)(=O)=O)c1)C(=O)Nc1nc(-c2cccc(N3C[C@@H](C)O[C@@H](C)C3)n2)cs1]
wherein the crystalline form is characterized by a powder x-ray diffraction pattern having peaks at 14.4 °2θ±0.2 °2θ and 17.4 °2θ±0.2 °2θ.

2. The crystalline form of claim 1, wherein the crystalline form is further characterized by a powder x-ray diffraction pattern having peaks at 11.7 °2θ±0.2 °2θ and 15.2 °2θ±0.2 °2θ.

3. The crystalline form of claim 1 or 2, wherein the crystalline form is further characterized by a powder x-ray diffraction pattern having peaks at 14.9 °2θ±0.2 °2θ and 18.5 °2θ±0.2 °2θ.

4. The crystalline form of any one of claims 1 to 3, wherein the crystalline form is further characterized by a powder x-ray diffraction pattern having peaks at 16.0 °2θ±0.2 °2θ and 19.1 °2θ±0.2 °2θ.

5. The crystalline form of any one of claims 1 to 4, wherein the crystalline form is further characterized by a powder x-ray diffraction pattern having a peak at 16.3 °2θ±0.2 °2θ.

6. The crystalline form of any one of claims 1 to 5, wherein the crystalline form is further characterized by a powder x-ray diffraction pattern having peaks at 20.6 °2θ±0.2 °2θ and 26.3 °2θ±0.2 °2θ.

7. The crystalline form of any one of claims 1 to 6, wherein the crystalline form is further characterized by a powder x-ray diffraction pattern having peaks at 21.2 °2θ±0.2 °2θ and 26.6 °2θ±0.2 °2θ.

8. The crystalline form of any one of claims 1 to 7, wherein the crystalline form is further characterized by a differential scanning calorimetry thermogram having an endothermic event onset at 173.5° C. to 180.1° C.

9. A crystalline form of the compound of formula (I): 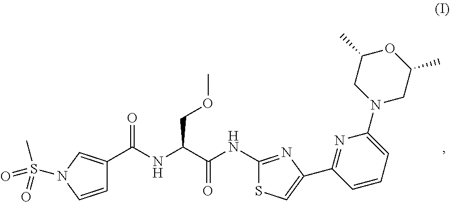 [SMILES: COC[C@H](NC(=O)c1ccn(S(C)(=O)=O)c1)C(=O)Nc1nc(-c2cccc(N3C[C@@H](C)O[C@@H](C)C3)n2)cs1]
wherein the crystalline form is characterized by a powder x-ray diffraction pattern having peaks at 7.7 °2θ±0.2 °2θ, 9.4 °2θ±0.2 °2θ and 17.0 °2θ±0.2 °2θ.

10. The crystalline form of claim 9, wherein the crystalline form is further characterized by a powder x-ray diffraction pattern having peaks at 18.8 °2θ±0.2 °2θ and 20.0 °2θ±0.2 °2θ.

11. The crystalline form of claim 9 or 10, wherein the crystalline form is further characterized by a powder x-ray diffraction pattern having a peak at 23.7 °2θ±0.2 °2θ.

12. The crystalline form of any one of claims 9 to 11, wherein the crystalline form is further characterized by a powder x-ray diffraction pattern having a peak at 14.8 °2θ±0.2 °2θ.

13. The crystalline form of any one of claims 9 to 12, wherein the crystalline form is further characterized by a differential scanning calorimetry thermogram having an endothermic event onset at 196.0° C. to 198.8° C.

14. A pharmaceutical composition comprising the crystalline form of any one of claims 1 to 13.

15. A unit dosage form comprising the crystalline form of any one of claims 9 to 13 or the pharmaceutical composition of claim 14.

16. The unit dosage form claim 15, wherein the unit dosage form is a capsule.

17. The unit dosage form of claim 15 or 16, wherein the unit dosage form comprises one or more of a filler, a disintegrant, a wetting agent, a glidant, a lubricant, and a capsule shell.

18. The unit dosage form of claim 17, wherein the filler is microcrystalline cellulose, mannitol, or a combination thereof; the disintegrant is croscarmellose sodium; the wetting agent is sodium lauryl sulfate; the glidant is colloidal silicon dioxide; and/or the lubricant is magnesium stearate.

19. The unit dosage form of claim 17 or 18, wherein the unit dosage form comprises 70 to 90% (w/w) of the filler.

20. The unit dosage form of claim 18 or 19, wherein the unit dosage form comprises 4 to 6% (w/w) of the disintegrant.

21. The unit dosage form of any one of claims 18 to 20, wherein the unit dosage form comprises 0.5 to 1.5% (w/w) of the wetting agent.

22. The unit dosage form of any one of claims 18 to 21, wherein the unit dosage form comprises 1.5 to 2.5% (w/w) of the glidant.

23. The unit dosage form of any one of claims 18 to 22, wherein the unit dosage form comprises 0.4 to 0.6% (w/w) of the lubricant.

24. The unit dosage form of any one of claims 18 to 23, wherein the capsule shell comprises a polymeric shell.

25. The unit dosage form of claim 24, wherein the polymeric shell comprises hypromellose and titanium dioxide.

26. A method of decreasing the activity of a BAF complex in a subject, the method comprising administering to the subject an effective amount of the crystalline form of any one of claims 1 to 13, the pharmaceutical composition of claim 14, or the unit dosage form of any one of claims 15 to 25.

27. A method of inhibiting BRM in a subject, the method comprising administering to the subject an effective amount of the crystalline form of any one of claims 1 to 13, the pharmaceutical composition of claim 14, or the unit dosage form of any one of claims 15 to 25.

28. A method of inhibiting BRG1 in a subject, the method comprising administering to the subject an effective amount of the crystalline form of any one of claims 1 to 13, the pharmaceutical composition of claim 14, or the unit dosage form of any one of claims 15 to 25.

29. A method of inhibiting BRM and BRG1 in a subject, the method comprising administering to the subject an effective amount of the crystalline form of any one of claims 1 to 13, the pharmaceutical composition of claim 14, or the unit dosage form of any one of claims 15 to 25.

30. A method of inducing apoptosis in a subject, the method comprising administering to the subject an effective amount of the crystalline form of any one of claims 1 to 13, the pharmaceutical composition of claim 14, or the unit dosage form of any one of claims 15 to 25.

31. The method of any one of claims 26 to 30, wherein the subject has cancer.

32. A method of treating a BAF complex-related disorder in a subject in need thereof, the method comprising administering to the subject an effective amount of the crystalline form of any one of claims 1 to 13, the pharmaceutical composition of claim 14, or the unit dosage form of any one of claims 15 to 25.

33. A method of treating a disorder related to a BRG1 loss of function mutation in a subject in need thereof, the method comprising administering to the subject an effective amount of the crystalline form of any one of claims 1 to 13, the pharmaceutical composition of claim 14, or the unit dosage form of any one of claims 15 to 25.

34. The method of claim 32 or 33, wherein the subject is determined to have a BRG1 loss of function disorder.

35. The method of any one of claims 32 to 34, wherein the BAF complex-related disorder or disorder related to a BRG1 loss of function mutation is a cancer, a viral infection, coffin siris, neurofibromatosis, or multiple meningioma.

36. The method of claim 35, wherein the neurofibromatosis is selected from the group consisting of neurofibromatosis type I (NF-1), neurofibromatosis type II (NF-2), and Schwannomatosis.

37. A method of treating cancer in a subject in need thereof, the method comprising administering to the subject an effective amount of the crystalline form of any one of claims 1 to 13, the pharmaceutical composition of claim 14, or the unit dosage form of any one of claims 15 to 25.

38. A method of reducing tumor growth of cancer in a subject in need thereof, the method comprising administering to the subject an effective amount of the crystalline form of any one of claims 1 to 13, the pharmaceutical composition of claim 14, or the unit dosage form of any one of claims 15 to 25.

39. A method of suppressing metastatic progression of cancer in a subject in need thereof, the method comprising administering to the subject an effective amount of the crystalline form of any one of claims 1 to 13, the pharmaceutical composition of claim 14, or the unit dosage form of any one of claims 15 to 25.

40. A method of suppressing metastatic colonization of cancer in a subject in need thereof, the method comprising administering to the subject an effective amount of the crystalline form of any one of claims 1 to 13, the pharmaceutical composition of claim 14, or the unit dosage form of any one of claims 15 to 25.

41. A method of reducing the level and/or activity of BRG1 and/or BRM in a cancer in a subject in need thereof, the method comprising administering to the subject an effective amount of the crystalline form of any one of claims 1 to 13, the pharmaceutical composition of claim 14, or the unit dosage form of any one of claims 15 to 25.

42. The method of any one of claims 37 to 41, wherein the cancer is non-small cell lung cancer, colorectal cancer, bladder cancer, cancer of unknown primary, glioma, breast cancer, melanoma, non-melanoma skin cancer, endometrial cancer, esophageal cancer, esophagogastric cancer, pancreatic cancer, hepatobiliary cancer, soft tissue sarcoma, ovarian cancer, head and neck cancer, renal cell carcinoma, bone cancer, non-Hodgkin lymphoma, small-cell lung cancer, prostate cancer, embryonal tumor, germ cell tumor, cervical cancer, thyroid cancer, salivary gland cancer, gastrointestinal neuroendocrine tumor, uterine sarcoma, gastrointestinal stromal tumor, CNS cancer, thymic tumor, adrenocortical carcinoma, appendiceal cancer, small bowel cancer, penile cancer, bone cancer, or hematologic cancer.

43. The method of claim 42, wherein the cancer is esophageal cancer.

44. The method of claim 42, wherein the cancer is non-small cell lung cancer, colorectal cancer, bladder cancer, cancer of unknown primary, glioma, breast cancer, melanoma, non-melanoma skin cancer, endometrial cancer, penile cancer, bone cancer, renal cell carcinoma, prostate cancer, or hematologic cancer.

45. The method of claim 44, wherein the cancer is non-small cell lung cancer.

46. The method of claim 44, wherein the cancer is melanoma, prostate cancer, breast cancer, bone cancer, renal cell carcinoma, or hematologic cancer.

47. The method of claim 46, wherein the cancer is melanoma.

48. The method of claim 47, wherein the melanoma is uveal melanoma, mucosal melanoma, or cutaneous melanoma.

49. The method of claim 48, wherein the melanoma is uveal melanoma.

50. The method of claim 46, wherein the cancer is prostate cancer.

51. The method of claim 46, wherein the cancer is hematologic cancer.

52. The method of claim 51, wherein the hematologic cancer is multiple myeloma, large cell lymphoma, acute T-cell leukemia, acute myeloid leukemia, myelodysplastic syndrome, immunoglobulin A lambda myeloma, diffuse mixed histiocytic and lymphocytic lymphoma, B-cell lymphoma, acute lymphoblastic leukemia, diffuse large cell lymphoma, or non-Hodgkin's lymphoma.

53. The method of claim 52, wherein the hematologic cancer is acute myeloid leukemia.

54. The method of claim 46, wherein the cancer is breast cancer.

55. The method of claim 54, wherein the breast cancer is an ER positive breast cancer, an ER negative breast cancer, triple positive breast cancer, or triple negative breast cancer.

56. The method of claim 46, wherein the cancer is a bone cancer.

57. The method of claim 56, wherein the bone cancer is Ewing's sarcoma.

58. The method of claim 46, wherein the cancer is a renal cell carcinoma.

59. The method of claim 58, wherein the renal cell carcinoma is a microphthalmia transcription factor family translocation renal cell carcinoma.

60. The method of any one of claims 37 to 41, wherein the cancer is non-small cell lung cancer, prostate cancer, esophagogastric cancer, hematologic cancer, or melanoma, wherein hematologic cancer is acute myeloid leukemia and melanoma is uveal melanoma.

61. The method of any one of claims 37 to 41, wherein the cancer is non-small cell lung cancer, prostate cancer, esophageal cancer, acute myeloid leukemia, or uveal melanoma.

62. The method of any one of claims 37 to 61, wherein the cancer expresses BRG1 and/or BRM protein.

63. The method of any one of claims 37 to 62, wherein the subject or cancer has a BRG1 loss of function mutation.

64. The method of claim 63, wherein the BRG1 loss of function mutation is in the ATPase catalytic domain of the protein.

65. The method of claim 63, wherein the BRG1 loss of function mutation is a deletion at the C-terminus of BRG1.

66. The method of any one of claims 37 to 65, wherein the cancer does not have, or has been determined not to have, an epidermal growth factor receptor mutation and/or an anaplastic lymphoma kinase driver mutation.

67. The method of any one of claims 37 to 66, wherein the cancer has, or has been determined to have, a KRAS mutation, a mutation in GNAQ, a mutation in GNA11, a mutation in PLCB4, a mutation in CYSLTR2, a mutation in BAP1, a mutation in SF3B1, a mutation in EIF1AX, a TFE3 translocation, a TFEB translocation, a MITF translocation, an EZH2 mutation, a SUZ12 mutation, and/or an EED mutation.

68. The method of any one of claims 37 to 67, wherein the cancer is metastatic.

69. The method of any one of claims 37 to 68, wherein the cancer is resistant to, or failed to respond to prior treatment with, an anticancer therapy.

70. The method of claim 69, wherein the anticancer therapy is a chemotherapeutic or cytotoxic agent, immunotherapy, surgery, radiotherapy, thermotherapy, or photocoagulation, or a combination thereof.

71. The method of claim 70, wherein the anticancer therapy is a chemotherapeutic or cytotoxic agent.

72. The method of claim 71, wherein the chemotherapeutic or cytotoxic agent is a mitogen-activated protein kinase (MEK) inhibitor and/or a protein kinase C (PKC) inhibitor.

73. The method of any one of claims 37 to 72, wherein the cancer is resistant to, or failed to respond to prior treatment with a PKC inhibitor.

74. The method of any one of claims 37 to 73, wherein the method further comprises administering to the subject an anticancer therapy.

75. The method of claim 74, wherein the anticancer therapy is a chemotherapeutic or cytotoxic agent, immunotherapy, surgery, radiotherapy, thermotherapy, or photocoagulation, or a combination thereof.

76. The method of claim 74 or 75, wherein the anticancer therapy is surgery, a MEK inhibitor, and/or a PKC inhibitor, or a combination thereof.

77. The method of claim 76, wherein the MEK inhibitor is selumetinib, binimetinib, or tametinib.

78. The method of claim 76, wherein the PKC inhibitor is sotrastaurin or IDE196.

79. A method for treating a viral infection in a subject in need thereof, the method comprising administering to the subject an effective amount of the crystalline form of any one of claims 1 to 13, the pharmaceutical composition of claim 14, or the unit dosage form of any one of claims 15 to 25.

80. The method of claim 79, wherein the viral infection is an infection with a virus of the Retroviridae family, a virus of the Hepadnaviridae family, a virus of the Flaviviridae family, a virus of the Adenoviridae family, a virus of the Herpesviridae family, a virus of the Papillomaviridae family, a virus of the Parvoviridae family, a virus of the Polyomaviridae family, a virus of the Paramyxoviridae family, or a virus of the Togaviridae family.

81. The method of any one of claims 26 to 80, wherein the effective amount of the crystalline form of the compound reduces the level and/or activity of BRG1 by at least 5% as compared to a reference.

82. The method of claim 81, wherein the effective amount of the crystalline form of the compound reduces the level and/or activity of BRG1 by at least 5% as compared to a reference for at least 12 hours.

83. The method of any one of claims 26 to 82, wherein the effective amount of the crystalline form of the compound reduces the level and/or activity of BRM by at least 5% as compared to a reference.

84. The method of claim 83, wherein the effective amount of the crystalline form of the compound reduces the level and/or activity of BRM by at least 5% as compared to a reference for at least 12 hours.
